(19) United States
(12) Patent Application Publication
(10) Pub. No.: US 2026/0301947 A1
(43) Pub. Date: Oct. 1, 2026

(54) MOTION-TRACKED IMMERSIVE FUNCTIONAL POSITRON EMISSION TOMOGRAPHY

(71) Applicant: Cornell University, Ithaca, NY (US)

(72) Inventors: Amirhossein Goldan, E. Setauket, NY (US); Mony Deleon, Ithaca, NY (US); Zipai Wang, Ithaca, NY (US)

(73) Assignee: Cornell University, Ithaca, NY (US)

(21) Appl. No.: 19/476,088

(22) PCT Filed: Apr. 19, 2024
(86) PCT No.: PCT/US2024/025431

§ 371 Date: Oct. 16, 2025

Related U.S. Application Data
(60) Provisional application No. 63/461,090, filed on Apr. 21, 2023.

(51) Int. Cl. G16H 50/20; A61B 5/00; A61B 5/16; A61B 6/03; A61B 6/46; A61B 6/50

(52) U.S. Cl. CPC G16H 50/20; A61B 5/16; A61B 5/6803; A61B 6/037; A61B 6/46; A61B 6/501

(57) ABSTRACT

Presented herein are directed to systems and methods for detecting conditions in subjects provided with stimuli. A computing system may send, to a headset situated on a head of a first subject in an upright posture, an instruction for the headset to present a stimuli to direct the subject to perform a task. The computing system may receive, via a tomography scanner arranged about the head of the first subject, a dataset defining a first plurality of tomograms of a first nervous system of the first subject. The computing system may obtain, via a sensor, a first signal associated with a first physiological function of the first subject over the time period. The computing system may determine, from the plurality of conditions, a condition associated with the first nervous system of the first subject based on applying a model to the first plurality of tomograms and the first signal.

### CROSS REFERENCES TO RELATED APPLICATIONS

The present application claims priority to U.S. Provisional Application No. 63/461,090, titled “Motion-Tracked Immersive Functional Positron Emission Tomography,” filed Apr. 21, 2023, which is incorporated herein by reference in its entirety.

### STATEMENT OF GOVERNMENT SUPPORT

The invention was made with government support under R01EB030413 awarded by the National Institute of Health (NIH). The government has certain rights to the invention.

### BACKGROUND

A tomographic device may scan a volume of a subject to generate a tomogram. A computing system may process the tomogram generated by the tomographic device to generate an output.

### SUMMARY

Aspects of the present disclosure are directed to systems and methods for detecting conditions in subjects provided with stimuli. One or more processors coupled with memory may send, to a headset situated on a head of a first subject in an upright posture, an instruction for the headset to present a stimuli to direct the subject to perform a task over a portion of a time period. The one or more processors may receive, via a tomography scanner arranged about the head of the first subject in the upright posture, a dataset defining a first plurality of tomograms of a first nervous system of the first subject over the time period. The one or more processors may obtain, via a sensor, a first signal associated with a first physiological function of the first subject over the time period, in at least partial concurrence of acquisition of the dataset defining the first plurality of tomograms via the tomography scanner. The one or more processors may apply a model to the first plurality of tomograms and the first response signal. The model may be established using a plurality of examples. Each of the plurality of examples may identify: (i) a respective plurality of second tomograms of a respective second nervous system of a respective second subject, (ii) a respective second signal associated with a respective second physiological function of the respective second subject, and (iii) a respective condition of a plurality of conditions identified for the respective second nervous system of the respective second subject. The one or more processors may determine, from the plurality of conditions, a condition associated with the first nervous system of the first subject based on applying the model to the first plurality of tomograms and the first signal. The one or more processors may store, using one or more data structures, an association between the condition and the first subject.

In some embodiments, the one or more processors may receive a plurality of user interactions by the subject while in the upright posture from an input/output (I/O) device in response to performing the task. The one or more processors may send a second instruction including feedback to provide to the subject, based on the task and plurality of interactions. The feedback may include at least one of a visual feedback, an auditory feedback, or a haptic feedback. In some embodiments, the one or more processors may generate an output based on at least one of the first plurality of tomograms, the first signal, or the association between the condition and the first subject. The one or more processors may provide, to a display for presentation, information using the output.

In some embodiments, the one or more processors may receive, from the sensor including a motion tracker situated on the headset, a plurality of coordinate measurements identifying motion of the head over the time period. The motion tracker may determine the plurality of coordinate measurements relative to an electromagnetic source within the tomography scanner. The one or more processors may generate the first plurality of tomographs based on the motion of head identified in the plurality of coordinate measurements and the dataset received via the tomography scanner. In some embodiments, the one or more processors may identify at least a portion of the first signal corresponding to the first plurality of tomographs, using a first plurality of timestamps of the first plurality of tomographs and a second plurality of timestamps of the first signal. The one or more processors may apply the model to the first plurality of tomographs and at least the portion of the first signal. The model may include at least one of a machine learning (ML) model or a statistical model.

In some embodiments, the one or more processors may send the instruction for the headset to present, via a display and a loudspeaker, an audiovisual stimuli to direct the first subject to perform the task including at least one of a cognitive task or a behavior task via an I/O device. The one or more processors may receive the dataset defining the first plurality of tomograms subsequent to injection of a hybrid bolus-infusion radiotracer into the first subject.

In some embodiments, the one or more processors may obtain, from the sensor including an eye tracker on the headset, the first signal to identify at least one of a saccade parameters or pupil size of eyes of the first subject over the time period. The one or more processors may determine the condition from the plurality of conditions associated with at least one of a dorsal raphe, a locus coeruleus, a superior colliculus, a basal ganglia, or a frontal cortex within the first nervous system of the first subject.

In some embodiments, the one or more processors may obtain, from the sensor including an electrocardiogram (ECG) sensor positioned on the first subject, the first signal including an ECG signal over the time period. The one or more processors may determine the condition from the plurality of conditions associated with at least one of a parasympathetic system in the first nervous system of the first subject. The plurality of conditions may include at least one of a cognitive function, an emotional regulation, or a stress management. In some embodiments, the one or more processors may obtain the first signal at least one of: (i) respiration sensor, (ii) an electroencephalogram (EEG) sensor, (iii) an electrodermal activity (EDA) sensor, or (iv) an electromyography (EMG) sensor. In some embodiments, the tomography scanner may include a body structure defining a scanning volume within which the head of the first subject is situated, wherein the tomography scanner is configured to permit the head of the first subject with a degree of movement within the scanning volume, wherein the tomography scanner is situated on a mobile structure.

### BRIEF DESCRIPTION OF THE DRAWINGS

FIG. 1: 3D model of the proposed upright MIf-PET platform with its 4 key features: 1) immersive augmented reality (AR) for multimodal behavior stimulation, 2) head/eye motion tracking, 3) heart-rate signal acquisition, and 4) upright fPET for multimodal quantification.

FIG. 2: Upright Prism-PET Scanner with EMMT: a) Schematic of the portable upright scanner with EMMT system which can be easily moved to any room or even placed inside a shielded imaging truck (to achieve high degree of diversity of subjects using a mobile platform). Note that a gooseneck arm may be used to move the EM source and place it in close proximity to the top of the head from the back of the scanner. b) Prism-PET super-module comprising of an array of 2×12 Prism-PET detector modules. c) Decagon gantry. d) A wearable sensor head-strap to position the sensor array at the top of the head.

FIG. 3: Prototype Prism-PET scanner with motion tracking: (a) Decagon-shaped conformal brain scanner. (b) Side-view of the scanner showing the front-end-module (FEM) boards without multiplexing. (c) The recently developed iMUX boards which have been developed.

FIG. 4: Experimental results of high-resolution PET imaging with rigid-body motion correction: Ultra-micro hot-spot and Hoffman brain phantom images using current state-of-the-art (a) and prototype Prism-PET (b) scanners. c) Comparison between TOF-DOI Prism-PET and Siemens biograph (zoomed with 1 mm voxels) in coronal view.

FIG. 5: Experimental results of High-Resolution Motion-compensated Reconstruction (HRMR) and eye tracking: a) New dynamic-HRMR or dHRMR framework to be iteratively developed and tested for Prism-PET to achieve DOI-corrected and motion compensated list-mode data (MC-LMD). b) Step motion tracking using EMMT (phantom is moved in steps using Thorlabs xyz-stage). EMMT system achieves the highest rotational and translational tracking accuracy. Inset shows the EMMT source and sensors. c) Prism-PET reconstructed images with 10 mm phantom motion, with and without motion correction. Also shown is the ground-truth schematic of both slices overlaid with the 2-mm LC regions colored in green. d) Results of eye tracking of a human subject using the ML headset showing successful relative measurement of the pupil diameter.

FIG. 6. a) Schematic of behavioral protocols of anti-saccade and oddball tasks. (a) The oculomotor paradigm design illustrates the sequence of prosaccade, anti-saccade, and oddball trail. (b) The experiment protocol depicts the structure composed of four blocks. (c) Picture of a participant wearing the MagicLeap2 AR google.

FIG. 7. a) Extracted 2D gaze locations using infrared red eye tracking cameras. The red curve represents the ground-truth stimulus marker position, and the blue curve indicates the detected 2D gaze position. b) Captured infrared eye images with white circles indicate the detected pupil.

FIG. 8. Anti-saccade experiment in block design. The red curve represents the ground-truth stimulus marker position, and the blue curve indicates the detected 2D gaze position.

FIG. 9. a) Example of the anti-saccade experiment, where the green dot represents the anti-saccade marker and appears randomly on the left or right side of the AR screen. b) Zoomed-in view of the gaze data, with anti-saccade reaction time and reaction amplitude indicated using arrows.

FIG. 10. Anti-saccade task without Go/No Go. a) Automatically detected saccade reaction rise indicated in black dash lines. b) Saccade reaction time vs. task number. c) boxplot of saccade reaction time for each module.

FIG. 11. Anti-saccade task with Go/No Go. a) Automatically detected saccade reaction rise indicated in black dash lines. b) Saccade reaction time vs. task number. c) boxplot of saccade reaction time for each module.

FIG. 12: a) ECG signal measured using ECG sensors, with detected R wave peaks indicated by red dots. b) Heart rate variability was extracted by calculating the R-R intervals. c) The power spectrum of the HRV signal, segmented into very low frequency (VLF), low frequency (LF), and high frequency (HF) energy bands.

FIG. 13: Schematic of oculomotor circuit highlighting areas involved in anti-and pro-saccade and pupil control (Adapted from Refs. and mostly based on animal neurophysiology). Note the convergence of cognitively relevant signals onto the small brainstem nuclei: 1) the locus coeruleus (LC)-norepinephrine (NE) neuromodulatory system, 2) the superior colliculus (SC).

FIG. 14: a) Cross-section view of the supine Prism-PET/EMMT scanner. b) Experimental results of a human subject's head motion correction using EMMT. c, d) Pictures of the portable Prism-PET scanner and portable crate to move or ship the scanner. e) Experimental results of phantoms comparing NeuroExplorer (NX) with Prism-PET.

FIG. 15: Upright Prism-PET/EMMT: a) Schematic of the portable upright scanner with EMMT system. Note that only a new upright adapter, gooseneck arm for the EM source, and portable cart need to be developed, and everything else (e.g., gantry, detectors, electronics) may be the same items that were developed and tested in Aim 1.1. b) A wearable sensor head-strap to fix the sensor array at the top of the subject's head in the sitting position. c) PETsys readout using 8 FEB/D and 2 DAQ boards and flowchart. D) Flowchart of new firmware and software data processing to be developed by PETsys to handle the large event rate expected of the large axial brain scanner.

FIG. 16 depicts a block diagram of a process for measuring brain functionality using a multi-dimensional, motion-tracked, immersive function PET (Mif-PET) platform, in accordance with an illustrative embodiment.

FIG. 17 depicts a block diagram of a system for detecting conditions in subjects provided with stimuli, in accordance with an illustrative embodiment;

FIG. 18 depicts a block diagram of a process of communicating with test environments in the system for detecting conditions in subjects provided with stimuli, in accordance with an illustrative embodiment;

FIG. 19 depicts a block diagram of a process of determining conditions of nervous systems of subjects in the system for detecting conditions in subjects provided with stimuli, in accordance with an illustrative embodiment;

FIG. 20 depicts a flow diagram of a method of detecting conditions in subjects provided with stimuli, in accordance with an illustrative embodiment; and

FIG. 21 depicts a block diagram of a server system and a client computer system in accordance with an illustrative embodiment.

### DETAILED DESCRIPTION

Following below are more detailed descriptions of various concepts related to, and embodiments of, systems and methods for detecting conditions in subjects provided with stimuli. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Section A describes a multimodal platform for motion-tracked immersive functional positron emission tomography.

Section B describes additional descriptions and results of the multimodal platform for motion-tracked immersive functional positron emission tomography.

Section C describes systems and methods for detecting conditions in subjects provided with multimodal stimuli using multimodal recordings.

Section D describes a network environment and computing environment that may be useful for practicing various computing-related embodiments described herein.

### A. Multimodal Platform for Motion-tracked Immersive Functional Positron Emission Tomography

The goal of the Motion-Tracked Immersive functional Positron Emission Tomography (MIf-PET) may be to develop a new multimodal technological platform with high temporal and spatial resolutions for behavioral neuroscience. The controlled, life-like augmented reality environment may be used with high ecological validity to capture complex behavioral patterns and quantify them in real time, using head and eye-tracking and functional positron emission tomography imaging of the brain, to understand aspects of behavior that can be ascribed to specific neuronal circuits.

A multimodal technological platform is proposed based on Motion-Tracked Immersive functional Positron Emission Tomography with high temporal and spatial resolution for capturing and quantifying complex behavior in the sitting position. For behavioral neuroscience, the controlled life-like augmented reality (AR) environment and its high ecological validity enable conducting novel multimodal sensory behavioral stimuli, which place unique demands on the brain and elicit different networks to respond in a very fine-grained manner.

Sensory stimulation includes visual information, auditory information, and haptic feedback. For multimodal measurement and analysis of behavior, the system tracks subjects' head and eye movements in real-time to help study the underlying construct or psychological phenomenon represented by that movement. Furthermore, during the motion-tracked immersive brain stimulation and recovery, it may utilize functional PET (fPET) using hybrid bolus-infusion injection-fluorodeoxyglucose (FDG) for accurate mapping of brain structures to function and behavior.

### 1. Platform

### 1.1. Toward Precision Behavioral Neuroscience Using a New Technological Platform for Synchronized Multimodal Sensory Stimulation and Multimodal Recording.

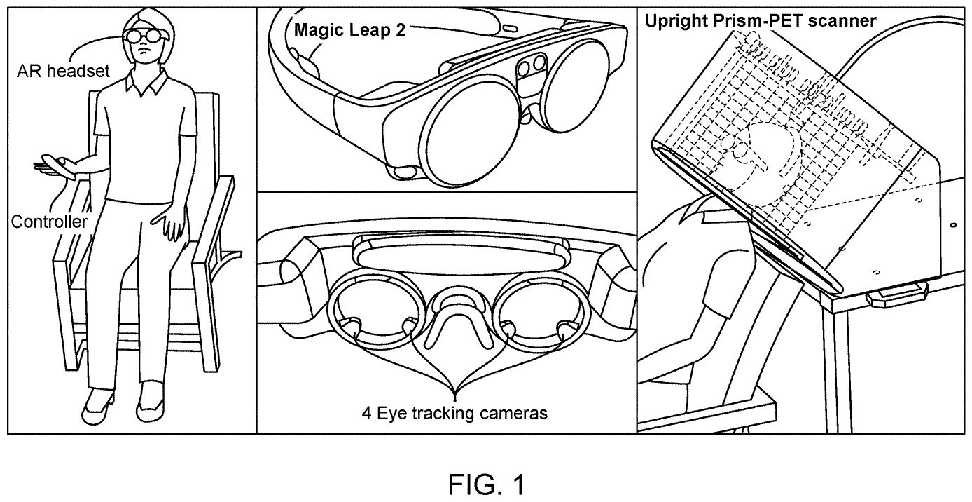

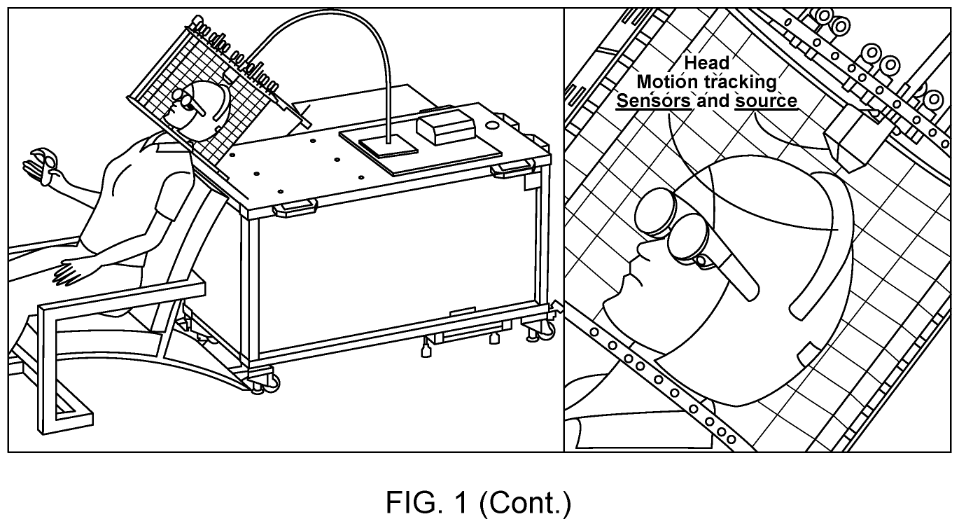

A multimodal technological platform is proposed based on Motion-tracked Immersive functional Positron Emission Tomography (MIf-PET) with high temporal and spatial resolution for capturing and quantifying complex behavior in the sitting position (FIG. 1). For behavioral neuroscience, the controlled, life-like augmented reality (AR) environment and its high ecological validity enables conducting novel multimodal sensory behavioral stimuli, which place unique demands on the brain and elicit different networks to respond in a very fine-grained manner. Sensory stimulation includes visual information, auditory information, and haptic feedback. For multimodal measurement and analysis of behavior, the system tracks subjects' head and eye movements in real-time to help study the underlying construct or psychological phenomenon represented by that movement. Furthermore, during the motion-tracked immersive brain stimulation and recovery, it may utilize functional PET (fPET) using hybrid bolus-infusion injection-fluorodeoxyglucose (FDG) for accurate mapping of brain structures to function and behavior.

To fully exploit the benefits of fPET and mitigate its limitations, an ultra-high sensitivity, ultra-high resolution, motion-compensated, portable, and upright Prism-PET brain scanner is proposed. What enables imaging in the sitting position using immersive behavioral tasks is the recently developed wearable electromagnetic motion tracker (EMMT) which is capable of achieving sub-millimeter motion tracking accuracy for both small (mm-scale) and large (cm-scale) head movements. EMMT, together with Prism-PET, can achieve the highest motion-compensated PET spatial resolution of ~1.2 mm. The Prism-PET/EMMT brain scanner may be synchronized with VR headsets for task-based dynamic neuroimaging. Furthermore, its high coincidence radiation sensitivity of 74 kcps/MBq (which is ~4 times higher than current state-of-the-art clinical PET-CT scanners) and ultra-high motion-compensated volumetric resolution of 1.7 μL (~20 times better than current state-of-the-art clinical PET-CT scanners) enable: 1) reconstruction of frames with time intervals as small as 1 s; and 2) functional mapping of small brain regions such as amygdala, hippocampus, and entorhinal cortex. High temporal and spatial resolution fPET (i.e., ~1 s frame time interval and ~1.2 mm resolution) may provide an unprecedented opportunity to quantify brain activation that can be potentially superior to functional magnetic resonance imaging (fMRI) because it's a fully quantitative index of neuronal activity and can be utilized in a more naturalistic setting by imaging subjects in an upright sitting position. Upright MIf-PET may provide novel tools to study changes in brain functional connectivity that may provide invaluable insights into these unique behavioral and cognitive processes in normal people and eventually in those with incipient brain disease.

### 1.2. Upright Prism-Pet Scanner and EMMT System.

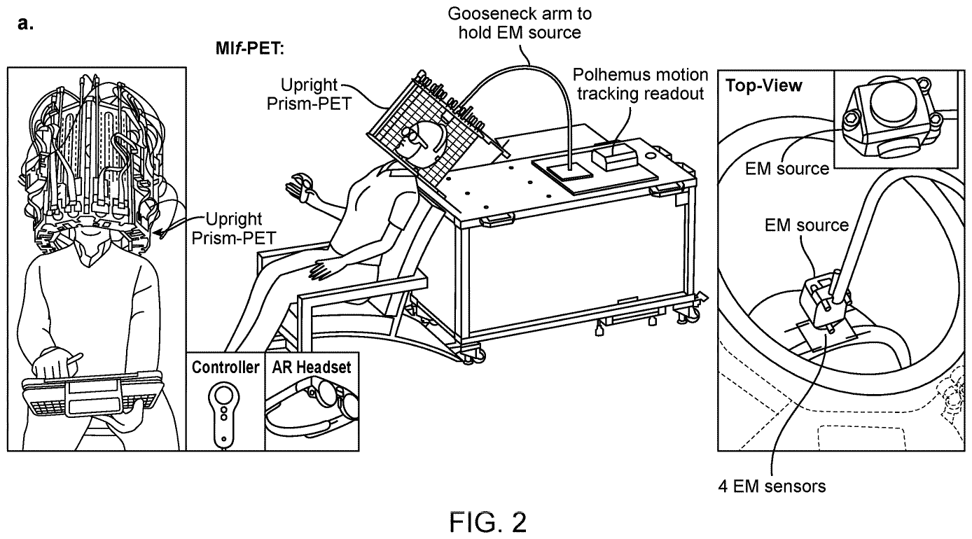

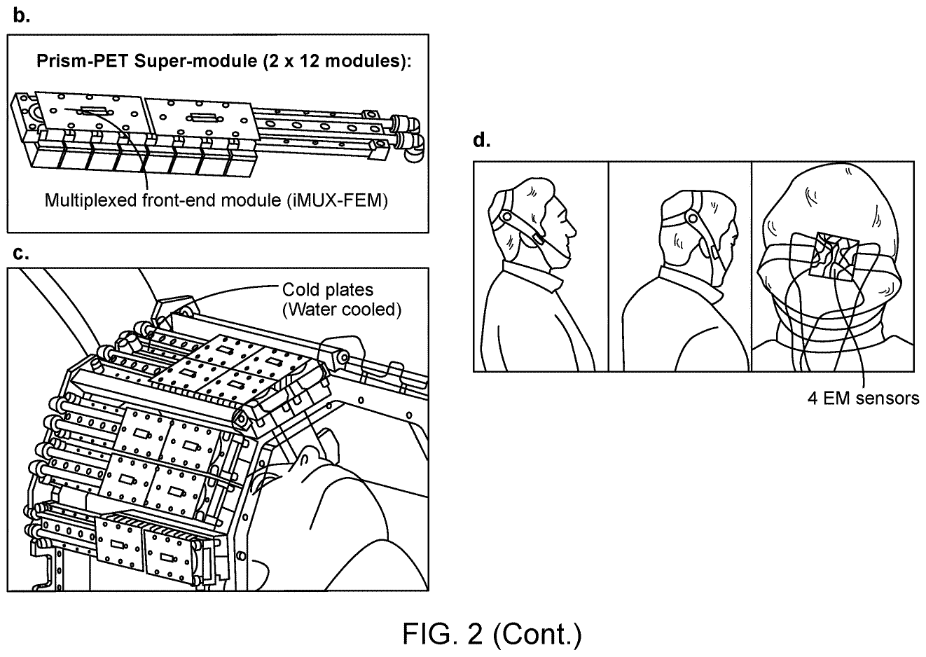

For Aim 2, the upright Prism-PET scanner and multi-sensor EMMT system (FIG. 2a) may be built. The upright gantry supports the full axial coverage with water-cooling and 12 rings of 4-to-1 coupled Prism-PET detector modules may be fabricated and characterized to achieve 33 cm axial FOV, and Prism-PET super-modules (FIG. 2b) may be precisely assembled around the conformal gantry. The chiller may be connected to the gantry cold plates (FIG. 2c), perform scanner calibration, and evaluate the system's temperature stability. Finally, the EMMT's coordinate system is aligned to that of the Prism-PET scanner using the 3 ²²Na radioactive sources which may be mounted on the EM source (see top-right inset in FIG. 2a). In this sub-aim, the upright Prism-PET gantry (with full axial coverage), cold plates, scanner cover, and portable cart are built to be able to move the scanner to any location and even inside a shielded imaging truck (to achieve a high degree of diversity of subjects using a mobile platform). The scanner's performance may be fully characterized using coincidence events. To evaluate the intrinsic spatial resolution, a ~1 MBq ²²Na point source (0.25 mm active diameter) may be scanned with 0.1 mm steps axially along the center of the scanner.

The intrinsic spatial resolutions of the detector pair may be derived from the FWHM values of the coincidence response function. The impact of the Compton recovery method on the spatial resolution may be investigated. Furthermore, using ²²Na and ⁶⁸Ge coincidence events, the coincidence timing resolution (CTR) may be measured with and without DOI corrections while using both classical and CNN-based approaches. For the construction of the EMMT system, a gooseneck arm (FIG. 2a) may be utilized so the EM source can be placed in close proximity to the top of the head where the 4 EM sensors are mounted (right inset in FIG. 2a). A wearable sensor head-strap may be worn by the subjects to position the 4 EM sensor array at the top of the head (FIG. 2d).

### 1.3. How Does the Proposed Platform Improve Behavioral Neuroscience Research?

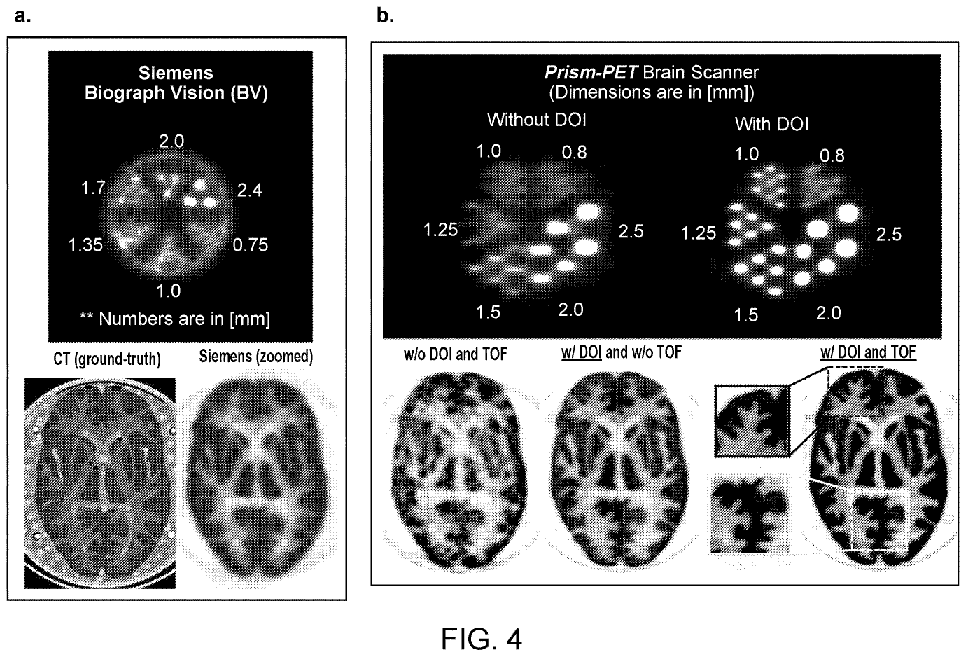

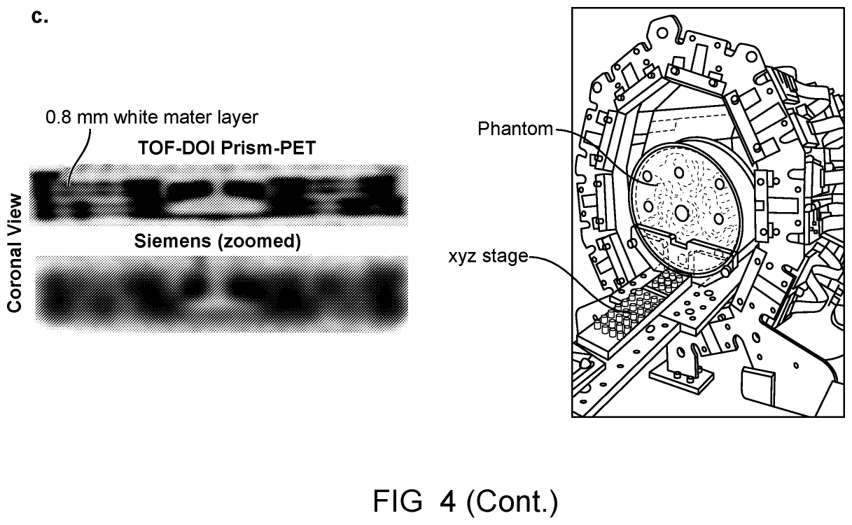

MIf-PET enables, for the first time, simultaneous multimodal brain stimulation and recording in the sitting position with high temporal and spatial resolutions. Prism-PET may have a significant clinical impact by enabling high signal-to-noise ratio (SNR) and high-resolution dynamic functional imaging with voxel-level kinetic modeling and reliable quantitative in vivo measurement of molecular targets in a host of small brain regions. Prism-PET, without head motion artifacts, enables accurate quantification in structures as small as 2 mm by substantially reducing the partial volume effect (PVE). However, uncorrected head motion, which can range from 1-5 mm for subjects lying down and without behavioral stimulation (and well beyond 5 mm for subjects in the sitting position and during AR-based behavioral tasks), blurs the image and substantially reduces the radiotracer recovery, which can, for example, lead to inaccurate parameter estimates in dynamic fPET. The diameter of a carotid artery ranges from 2-5 mm, and FIG. 4b shows how the effects of PVE and motion together may substantially reduce the accuracy of PET to quantify radiotracer kinetics in those regions for obtaining an accurate image-derived input function (IDIF). FIG. 4b also shows how high-resolution motion-compensated reconstruction (HRMR), using both Prism-PET and EMMT, can substantially reduce both PVE and spatial blur due to head motion and achieve high quantification accuracy for obtaining IDIF compared to the ground-truth arterial input function (AIF).

### 2. Methods

The MIf-PET platform employs three technological innovations for behavioral neuroscience: (1) multimodal sensory stimuli using a compact and light-weight AR headset to achieve high ecological validity and enhance both task engagement and task compliance; (2) submillimeter-accurate head motion capture (using the recently developed EMMT) and eye movement tracking (using four eye tracking cameras) as behavioral measurement tools since, for example, it has been confirmed that for subjects with psychopathologies, eye movement is associated with the cognitive and motor system; (3) accurate heart-rate measuring using 3-node portable ECG sensors; and (4) ultra-high sensitivity and ultra-high resolution motion-compensated upright fPET neuroimaging using the recently developed Prism-PET brain scanner. All of these tools have a temporal resolution of ~1 s and may be synchronized to capture and quantify behavior in a more naturalistic sitting position for enhanced task engagement.

### 3. Preliminary Results

### 3.1. Development and Testing of the Motion Compensated Upright Prism-Pet Brain Scanner.

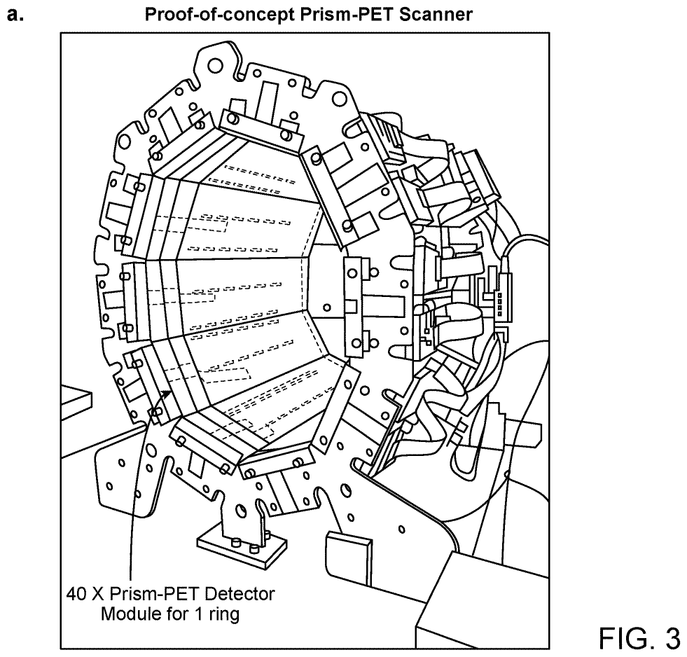

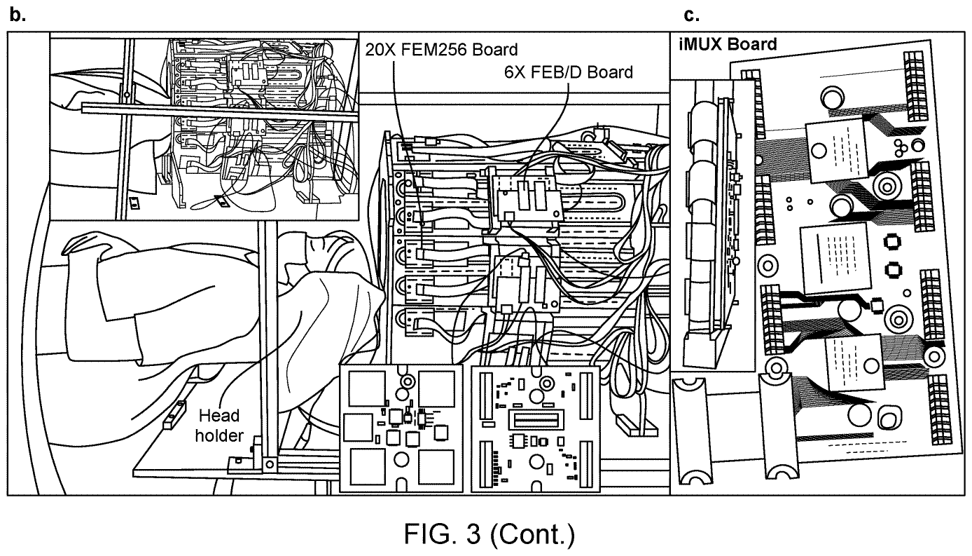

Rationale: To fully exploit the benefits of fPET and mitigate its limitations (i.e., increase fPET sensitivity by reducing frame time interval, eliminate motion artifacts, and use high-resolution PET imaging to enable analysis of brain activation in small regions), an ultra-high sensitivity, ultra-high resolution, motion-compensated, portable, and upright Prism-PET brain scanner (a recently built prototype is shown in FIG. 3) is proposed. What enables imaging in the sitting position using immersive behavioral tasks is the recently developed wearable electromagnetic motion tracker (EMMT) which is capable of achieving sub-millimeter motion tracking accuracy for both small (mm-scale) and large (cm-scale) head movements. The upright Prism-PET brain scanner, with a conformal decagon geometry and 33 cm of axial coverage, may be built. The electromagnetic motion tracking (EMMT) system may be integrated and synchronized to the Prism-PET list-mode data (PET-LMD) and EMMT list-mode data (EMMT-LMD) to achieve ultra-high-resolution motion-compensated PET images. The compact design maximizes scanner geometrical sensitivity and minimizes the number of detector modules per ring and scanner cost. The excellent depth-of-interaction (DOI) localization and motion tracking accuracy of the Prism-PET detector modules may substantially reduce parallax effect (PE) and yield uniform ~1.2 mm spatial resolution throughout the entire FOV.

Rigor: The 4-to-1 coupled Prism-PET detector module has 16×16 scintillators, each 1.5×1.5×30 mm³, directly glued to an 8×8 array of SiPMs with pixel size of 3×3 mm². The scanner may be built using the following steps: 1) Fabricate the remaining Prism-PET modules using procedures established in the preliminary work to extend the axial FOV (AFOV) to 12 rings of detectors (or 33 cm AFOV) with tapered crystal columns; 2) Precision assembly of modules around the gantry using procedures; 3) connect all detector blocks to interleaved multiplexing (iMUX) front-end modules (iMUX-FEM); 4) connect iMUX-FEM to the digital PETsys FEB/D readout system and clock/trigger synchronization boards to collect hits and transmit assembled frames to the data acquisition (DAQ) board which merges all FEB-D frames and transmits the assembled coincidence list-mode data to the computer; 6) use a gooseneck arm to bring the EMMT source inside the gantry from the back of the scanner and position it close to the EMMT sensors, which may be mounted at the top of the subject's head; 7) optimize the wearable head strap that holds 4 EMMT micro-sensors at the top of the human head for real-time monitoring of head motion; 8) calibrate the EMMT system and align its coordinate system with that of the Prism-PET scanner; and 9) finally, iteratively develop and refine the dynamic high-resolution motion-compensated reconstruction (dHRMR) using both PET-LMD and EMMT-LMD.

### 3.2. Reconstructed Phantom Images Using the Prototype Prism-PET Scanner.

Ultra-micro hot-spot and 2D Hoffman brain phantom images were reconstructed using the state-of-the-art Siemens Biograph Vision (FIG. 4a) and prototype high-resolution Prism-PET scanners (FIG. 4b) with the latter showing ultra-high-resolution performance using the developed TOF-DOI reconstruction framework. Note that phantoms were filled with 1 mCi of 18F activity. The world's highest-resolution PET phantom images show perfect reconstruction of 1-mm-diameter hot rods using a human-scale prototype brain scanner (top-right in FIG. 4b).A comparison between TOF-DOI Prism-PET and zoomed Siemens Biograph mCT reconstruction in coronal view (FIG. 4c) shows how Prism-PET enables, for the first time, clear visualization of the 0.8 mm white matter layers.

3.3. Experimental Results of Prototype Conformal Prism-PET Scanner (Single Ring of Modules) with Real-Time Tracking of Head And Eye Movements.

For high-resolution brain PET imaging, both the position and orientation of the head must be precisely estimated in real time. Recent motion tracking technologies use infrared optical systems to construct the 3D profile of an object along the camera's line-of-sight (e.g., human subject's face) and track motion using pattern recognition. However, such optical systems are complex, expensive, and prone to occlusion (especially in small-diameter brain PET scanners). For the first time, a cost-effective, non-optical, and wearable electromagnetic motion tracking (EMMT) system, without line-of-sight restrictions, is proposed that, with careful design and engineering, may overcome the electric and magnetic interferences and achieve precise six degree-of-freedom (DOF) motion capture at a sampling frequency exceeding 240 Hz.

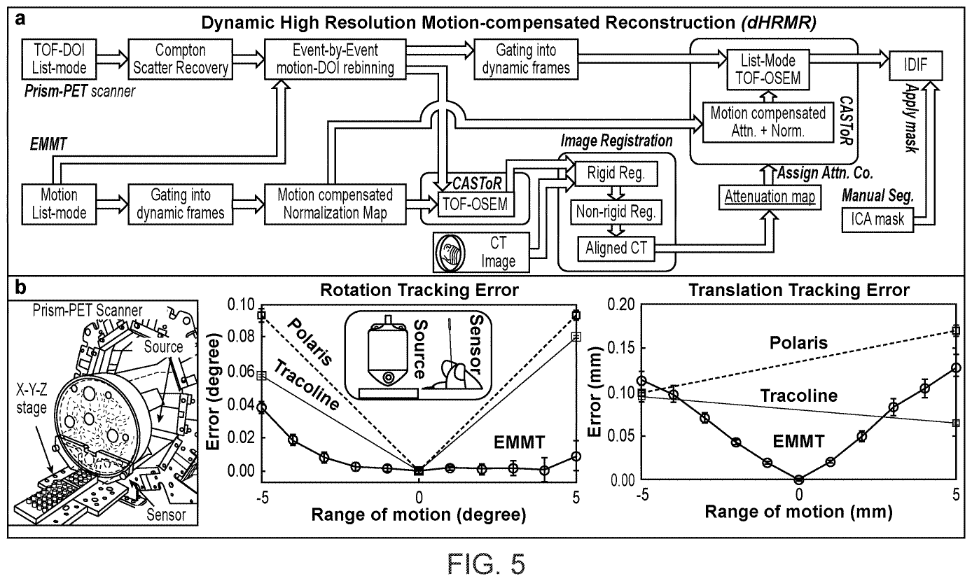

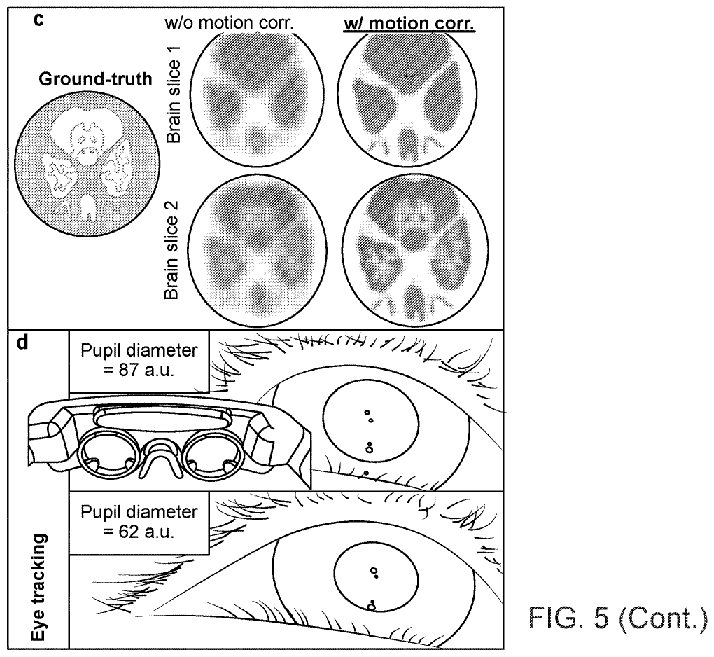

The flowchart for the dynamic high-resolution motion-compensated reconstruction (dHRMR) is shown in FIG. 5a. As a prototype, an EMMT system was built for tracking phantoms with step motion, as shown in FIG. 5b, using an electromagnetic (EM) source and multiple EM micro-sensors. The EMMT has been calibrated to align its coordinate system with that of the Prism-PET scanner. The highest rotational and translational tracking accuracy has been achieved and compared to optical Polaris and Tracoline systems (FIG. 5b). Reconstructed TOF-DOI Prism-PET images, without motion correction (left column in FIG. 5c), show poor spatial resolution with unresolvable locus coeruleus (LC) regions due to severe motion-induced blur. However, using the HRMR framework of FIG. 5a, resulted in being able to successfully recover the high-resolution performance of TOF-DOI Prism-PET scanner with a clear visualization of both LC hot-spot regions (right column in FIG. 5c). The world's highest-resolution PET phantom images show perfect reconstruction of 1-mm-diameter hot rods using a human-scale prototype brain scanner (top-right in FIG. 4b). Finally, using the ML2's 4 eye tracking cameras, the relative size (i.e., diameter) and the location of the pupil (FIG. 5d) have been measured.

### 3.4. Development of Eye Tracking Using ML2.

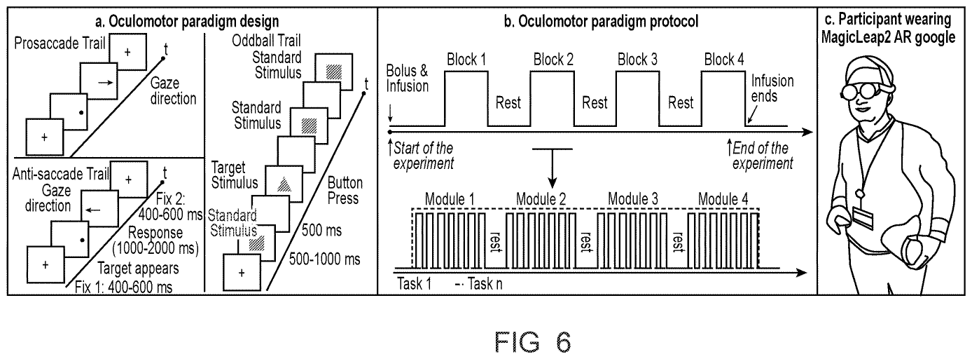

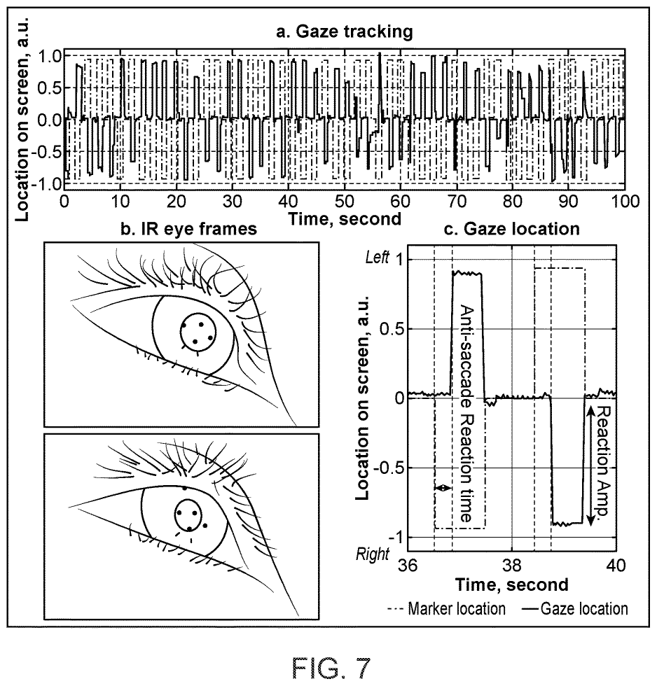

During the scan, they will complete saccade tasks and oddball tasks (FIGS. 6a and b). Each block will progressively increase in difficulty to impose varying cognitive load levels. The saccade task starts with a fixation cross displayed at the center of the virtual screen of the AR glass. The color of the cross cues the participants to execute a prosaccade (looking towards a target) or an anti-saccade (looking away from the target in the opposite direction) (FIG. 6a). The sequence of saccade types and directions will be randomized. Participants may be asked to hold their gaze in the position until the target disappears. In the oddball experiments, the participants may be presented with two visual stimuli: a blue rectangle as the standard stimulus, and a blue triangle as the target stimulus (FIG. 6a). The AR device (e.g., ML2) may be synchronized with the PET scanner and record the 3D eye movement using the four infrared eye-tracking cameras (two cameras per eye). The pupil, which appears dark in the infrared-illuminated eye camera image, may be automatically segmented and detected (FIG. 7b).

Calibrations using screen markers may be carried out to obtain the transfer functions that can map the pupil positions to the scene space (obtained by world cameras). The velocity of eye movements may be calculated from the eye positions, and saccade movements with an eye position shifted larger than two degrees from the steady baseline may be detected (FIG. 7).

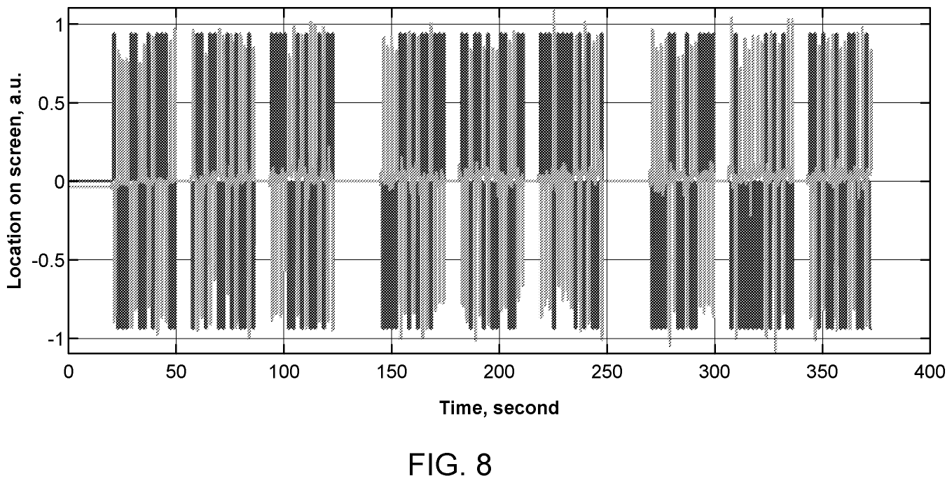

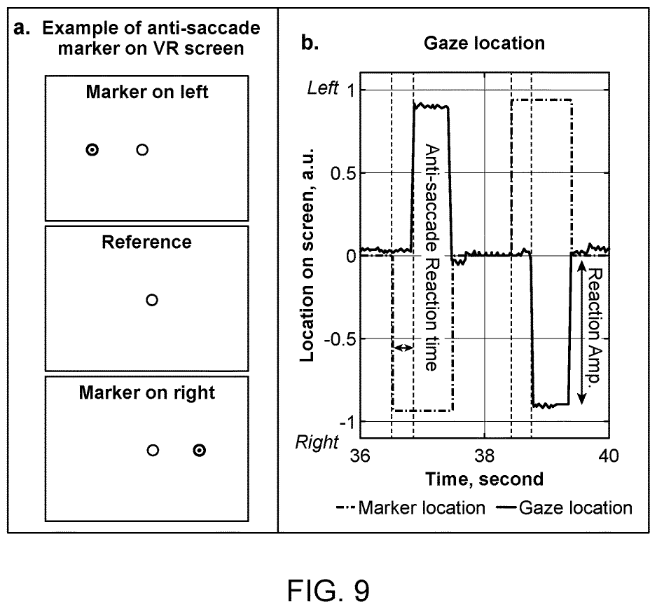

Since a minimum of 90 ms is required for a visual signal to propagate through the oculomotor system and trigger a saccade, saccades occurring 90-800 ms post-stimulus were grouped into task-relevant responses, and those occurring below 90 ms were classified as anticipatory saccades. For the viable saccades, the reaction time, velocity, amplitude (degree), disengagement rate, and direction errors were calculated (FIGS. 8 and 9).

Eye movements have the potential to serve as a sensitive marker for cognitive changes and deterioration. The eye blink rate (EBR), on the other hand, is positively correlated with central dopamine activity, and it can reliably predict individual differences in performance on many cognitive tasks. In AD, the unique pattern of neurodegeneration may affect the neural circuitry of the eye movement system, and eye-tracking studies could help differentiate AD from other cognitive disorders. It has also been shown that people with early-stage AD could have mild impairments in cognitive inhibition that can be detected by eye-tracking paradigms.

### 3.5. Heart Rate Measurement.

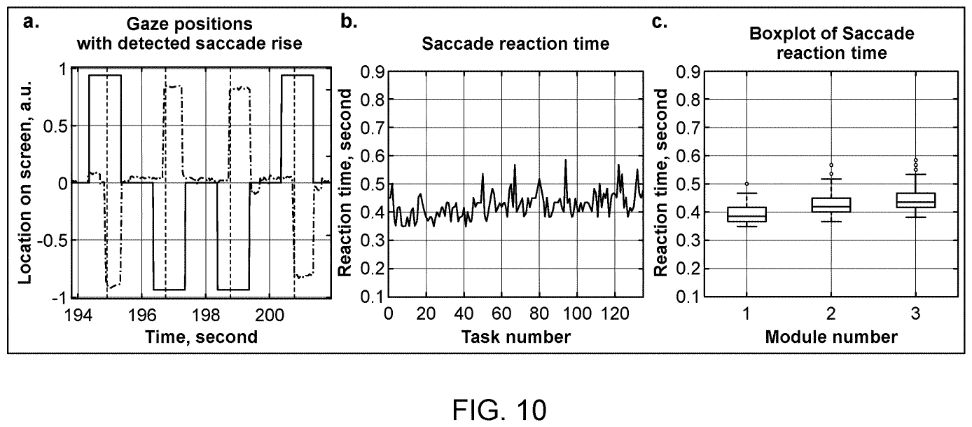

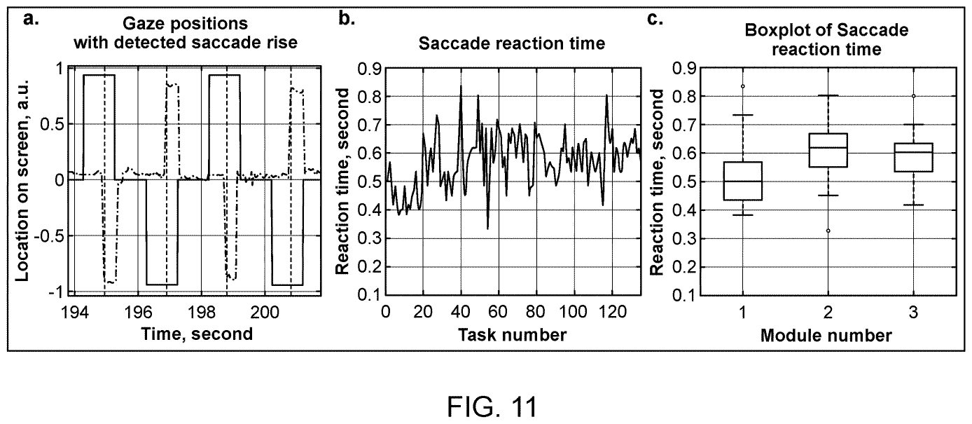

Heart rate and heart rate variability are indicators of autonomic nervous system functions. The HRV may provide an index of the parasympathetic nervous system, which is linked to a range of psychophysiological processes, including cognitive functions, emotional regulation, and the management of stress. The ECG signal was measured using a high-resolution, three-lead portable ECG sensor (FIG. 10a). The R-peaks in the ECG data were detected, and the RR interval between each peak was calculated to generate the HRV curve (FIG. 10b.). The analysis of HRV was focused on both time-domain and frequency-domain measures. Time domain analysis includes metrics like the standard deviation of RR intervals (SDNN) and the percentage of consecutive RR intervals over 50 ms (pNN 50). The RR intervals were also transformed into the frequency domain, and its power spectrum may be segmented into three frequency bands: very low frequency (VLF; 0.0037-0.04 Hz), low-frequency band (LF; 0.04-0.15 Hz), and high-frequency band (HF, 0.15-0.4 HZ) (FIG. 10c). The energy ratio between LF and HF and other ECG parameters enabled evaluation of the focus and stress levels of the participants, which provided information on task difficulty, task engagement, and attention level from another dimension.

### B. Additional Description and Results of the Multimodal Platform for Motion-Tracked Immersive Functional Positron Emission Tomography

### 1. Introduction

For precision behavioral neuroscience research, a novel technological platform based on Motion-tracked Immersive functional Positron Emission Tomography (MIf-PET), with high temporal and spatial resolution (i.e., ~1 s frame time interval and ~1 mm resolution) for multimodal stimulation and multimodal quantification of complex behavior in the more naturalistic sitting position (FIG. 1), is proposed. Additionally, this can track a subject's head, hand, eye movements, and heart rate in real time and study the underlying construct or psychological processes. Brain stimulation using the controlled, life-like augmented reality (AR) environment and its high ecological validity enables conducting of novel behavioral paradigms that place unique demands on the brain and elicit different networks to respond in a very fine-grained manner. Such brain activation is crucial as it amplifies the subtle variations in brain functions, especially in small brain regions, and captures more behaviorally relevant information than resting-state functional connectivity. For example, eye tracking during pro-and anti-saccade paradigms (with real-time tracking of saccade parameters) is a non-invasive and sensitive way to assess: 1) altered patterns of oculomotor behavior, and consequently 2) subtle cognitive decline in subjects with mild cognitive impairment (MCI) and patients with Alzheimer's disease (AD).

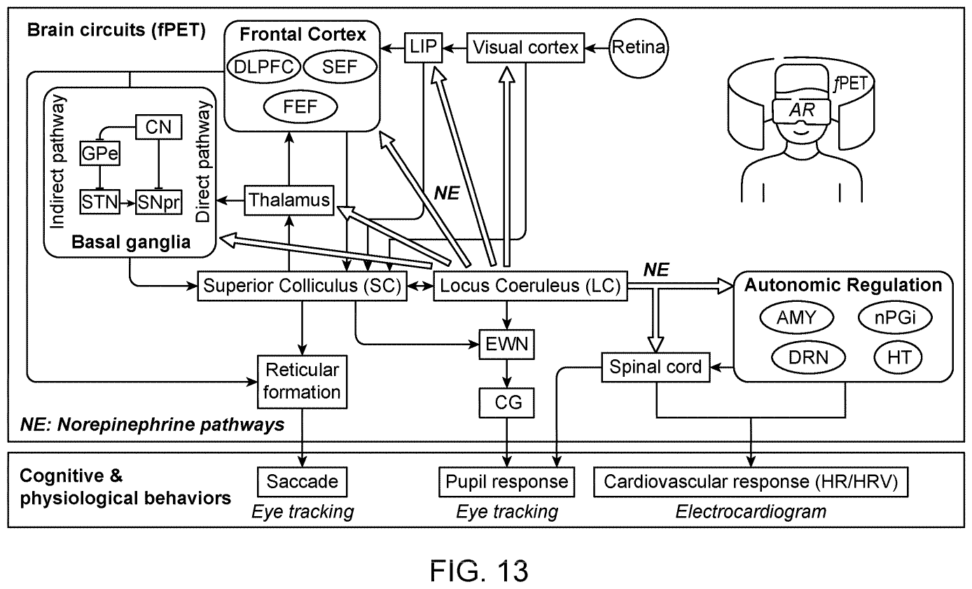

In addition to tracking saccade parameters, the real-time eye tracking AR headset's variable background luminance level and eye images were captured at 60 Hz using the four infrared eye tracking cameras to precisely quantify the dynamic of task-evoked pupillary responses (TEPR) during non-luminance-mediated and luminance-mediated generation of reflexive and voluntary saccades. It is suggested that pupil size is a window on neural mechanisms underlying cognition and have identified the convergence of cognitively relevant signals onto the two brainstem nuclei: 1) the locus coeruleus (LC)-norepinephrine (NE) neuromodulatory system, and 2) the superior colliculus (SC), specifically the intermediate layers (FIG. 13). However, saccade parameters and pupil size alone are not enough and may be combined with simultaneous measurement of neural activity in multiple brain regions. That is why the proposed technological platform also allows task-evoked functional PET (fPET) imaging using a hybrid bolus-infusion injection of a radiotracer. MIf-PET aims to enable in vivo characterization of these kinds of circuit detail in the sitting position and without head confinement, and provide a solid basis for designing immersive behavioral tasks to better understand functional neural activity.

Aim 1, (n=65): Optimization of AR-based oculomotor behavioral tasks and synchronization with hybrid bolus-infusion fPET in the supine position. Cognitive correlates of oculomotor behavior for AD research may be investigated during the execution of prosaccades towards, or anti-saccades away from, immersive stimuli with varying cognitive load (and integrated with a two-stimulus oddball paradigm) while Saccade parameters, Pupil size, and Heart rate variability (or SPH) are being recorded. The AR-based behavioral tasks and head-motion-compensated dynamic fPET imaging protocol using n=5 healthy control (HC) subjects may be optimized. N=60 subjects over years 1-3 (20 HC, 20 MCI, and 20 AD) may be recruited, and task-evoked recording of SPH with simultaneous fPET neuroimaging using clinical FDG PET-CT in the supine position may be performed.

Aim 2: Development and testing of the ultra-high resolution and upright Prism-PET/EMMT brain scanner. To fully exploit the benefits of fPET, an ultra-high resolution, high sensitivity, motion-compensated, portable, and upright Prism-PET brain scanner (32 cm axial field-of-view, 74 kcps/MBq sensitivity, 1 mm spatial resolution) may be built. What enables imaging in the sitting position using immersive behavioral tasks is the recently developed wearable electromagnetic motion tracking (EMMT) system, which is capable of achieving sub-millimeter motion tracking accuracy.

Aim 3, (n=45): Immersive task-evoked SPH tracking with simultaneous upright fPET to study the link between oculomotor behavior and the neural circuitry of cognition. N=45 subjects over years 4-5, aged 60-90 years (15 HC, 15 MCI, 15 AD), may be recruited. While seated in the upright position (and without head confinement), subjects may perform the optimized immersive oculomotor behavioral tasks using the AR headset (developed in aim 1) with simultaneous SPH recording (developed in aim 1), and functional neuroimaging using the optimized fPET imaging protocol (developed in aim 1), and the ultra-high resolution and upright Prism-PET/EMMT scanner (developed in aim 2). Static metabolic activity related to saccadic eye movements and the dynamics of pupil dilation may be calculated using summed PET images. Dynamic fPET images may be used to (a) calculate activation maps for anti-saccade, prosaccade, and pupil dilation maps separately (not feasible using traditional static PET) and (b) calculate ultra-high resolution metabolic connectomes differentiating anti-saccade, prosaccade, and pupil responses within the oculomotor network in healthy and pathological conditions.

### 2. Impact

### 2.1. Context.

Humans interact with and perceive the world in an upright orientation. Neuroscience research has well established that body orientation alters cerebral blood flow, the amount of cerebrospinal fluid in the brain, intracranial pressure, cell-type-specific firing rates, and resting-state functional connectivity. Electroencephalogram (EEG) studies have shown that the supine position, compared to upright, leads to decreased resting-state cortical activity. Moreover, posture plays an important role in modulating autonomic nervous system activation and cognitive functions in the supine position are more susceptible to factors such as sleep quality and chronic stress levels. Sleep quality strongly affects the reaction time of the working memory test when participants are in the supine position but has no influence on performance when they are in the upright position.

### 2.2 Implications With Neuroscience.

Neuroscience research is almost exclusively based on functional neuroimaging techniques and synchronized task executions that are conducted in a supine position while subjects remain motionless and adhere to non-ecological comportments within a confined space. Although some upright magnetic resonance imaging (MRI) scanners are commercially available for seated or standing subjects, they are based on low magnetic fields, which result in very poor functional MRI (fMRI) sequencing and spatial resolution. Acoustic noise from MRI scanners also impacts resting-state brain activity, and magnetoencephalography (MEG) studies found that it may consistently reduce the connectivity of cortical networks (especially for the right auditory and sensor-motor networks). Given the influence of posture on brain activity and behavior, the transferability and generalizability of many neuroimaging investigations may be undermined because of the inconsistency between ecological postures and imaging constraints (and, in the case of MRI, the scanner's acoustic noise).

### 2.3. Multimodal Sensory Stimuli and Multimodal Recording Approach.

Toward precision behavioral neuroscience using this new technological platform for synchronized multimodal sensory stimulation and multimodal recording in an upright sitting position and without head confinement. For precision behavioral neuroscience research, a novel technological platform based on Motion-tracked Immersive functional Positron Emission Tomography (MIf-PET) with high temporal and spatial resolution (i.e., ~1 s frame time interval and ~1 mm resolution) for multimodal stimulation and multimodal quantification of complex behavior in the more naturalistic upright sitting position is proposed.

### 2.3.1. Immersive Augmented Reality (AR).

For behavioral neuroscience, the controlled, life-like augmented reality (AR) environment and high ecological validity enable the conduct of novel multimodal sensory behavioral stimuli that place unique demands on the brain and elicit different networks to respond in a very fine-grained manner. Sensory stimulation includes visual information, auditory information, and haptic feedback, which provide the immersed AR user with a more natural and ecological interaction with their enhanced surroundings.

### 2.3.2. Tracking of Head Motion, Eye Movement, and Heart Rate.

For multimodal measurement and analysis of behavior, the system tracks a subject's head, eye, and heart rate in real time to help study the underlying construct or psychological phenomenon represented by that movement. What enables imaging in the upright sitting position using immersive behavioral tasks (with free head movement) is the recently developed wearable electromagnetic motion tracking (EMMT) which is capable of achieving sub-millimeter motion tracking accuracy for both small (mm-scale) and large (cm-scale) head movements. The four infrared eye tracking cameras mounted on the AR headset may be used to measure oculomotor parameters such as task disengagement, saccadic reaction time, mean correct prosaccade velocity and amplitude, etc. Oculomotor is linked to cognitive measures and alterations in disease and oculomotor tasks can generate a wealth of behavioral biomarkers for neurodegenerative diseases. Finally, a low-noise 3-electrode electrocardiogram (ECG) sensor may be used to measure heart rate and heart rate variability (HRV) as an index of autonomic control of the heart. The different levels of autonomic activation contribute to differences in cognitive load, mental fatigue, and sleepiness and can be used quantify subjects' task disengagement.

2.3.3. Motion-Compensated Functional Pet (fPET) in Upright Orientation.

During immersive brain stimulation and recovery, fPET using hybrid bolus-infusion injection of fluorodeoxyglucose (FDG) for accurate mapping of brain structures to function and behavior may be utilized. To fully exploit the benefits of fPET and mitigate its limitations, an ultra-high sensitivity, ultra-high resolution, motion-compensated, portable, and upright Prism-PET brain scanner is proposed. EMMT together with Prism-PET can achieve the highest motion-compensated PET spatial resolution of ~1 mm. The Prism-PET/EMMT brain scanner may be synchronized with AR headsets for task-based dynamic neuroimaging. Furthermore, its high coincidence radiation sensitivity of 74 kcps/MBq (which is ~4 times higher than current state-of-the-art clinical PET-CT scanners) and ultra-high motion-compensated volumetric resolution of 1.7 μL (~20 times better than current state-of-the-art clinical PET-CT scanners) enable 1) reconstruction of frames with time intervals as small as 1 s and 2) functional mapping of small brain regions (such superior colliculus) to enable accurate mapping of, for example, oculomotor brainstem circuitry.

### 2.4. Clinical Translation:

Distinctive oculomotor behaviors, specifically cognitive correlates of anti-saccade behavior, in AD may be investigated. Studies have shown that people with early-stage AD could have mild impairments in cognitive inhibition that can be detected by eye-tracking paradigms.

More specifically, patients demonstrate abnormally high levels of saccadic distractibility when carrying out the anti-saccade tasks. The stimuli and tasks may be designed to test saccade prediction techniques using AR headsets with eye trackers. Measures of autonomic activation (including heart rate and HRV) and oculomotor function (including correct and incorrect anti-saccades and prosaccades, saccadic reaction time, anticipatory saccades, directional errors, and saccade gain) may be included during anti-saccade, no-saccade, prosaccade and smooth pursuit tasks. The behavioral tasks may be synchronized with fPET neuroimaging to study oculomotor and disease-impaired circuitry and calculate brain activation maps for anti-saccade and prosaccade tasks. It is hypothesized that the high temporal and spatial resolutions multimodal MIf-PET in the upright sitting position may allow disentanglement of metabolic signals related to anti-saccades, prosaccades, and directional errors (failure of inhibitory control) for the first time.

### 2.4.1. Heterogeneity

FDG-PET biomarkers may be performed to establish at resting-state for AD onset and progression based on functional connectivity. However, these existing studies demonstrate a lack of reproducibility, primarily caused by sample heterogeneity, which has made it difficult to establish clinically relevant early-stage PET biomarkers for AD. This heterogeneity may be reduced with the proposed multi-modal MIf-PET platform by establishing a robust behavioral-functional-molecular biomarker using immersive oculomotor paradigms with eye tracking such as prosaccade and anti-saccade tasks. Saccade task performance deficits have been correlated with AD, and relative performance has even been correlated with dementia severity. Task-based FDG-fPET has been shown to have high test-retest reliability using oculomotor tasks. Thus, the proposed immersive behavioral tasks synchronized with the ultra-high resolution fPET in the more naturalistic upright orientation would be a strong candidate to precisely and reproducibly probe functional connectivity pathways in HC, MCI, and AD while reducing the effects of sample heterogeneity.

### 3. Implications

The MIf-PET platform employs three technological innovations for behavioral neuroscience: (1) multimodal sensory stimuli using a compact and light-weight AR headset to achieve high ecological validity and enhance both task engagement and task compliance; (2) submillimeter-accurate head and arm motion capture (using the recently developed EMMT), eye movement tracking (including saccade parameters and pupil size using 4 eye tracking cameras), and heart rate tracking as behavioral measurement tools; (3) ultra-high sensitivity and ultra-high resolution motion-compensated upright fPET neuroimaging using the recently developed Prism-PET brain scanner to allow mapping of very small brain regions such as SC and LC. All of these tools have a temporal resolution of ~1 s or better and may be synchronized to capture and quantify behavior in a more naturalistic sitting position for enhanced task engagement.

### 4. Methods

4.1. Aim 1, (n=65): Optimization of AR-Based Oculomotor Behavioral Tasks and Synchronization with Hybrid Bolus-Infusion fPET in the Supine Position.

Brain stimulation using the controlled life-like augmented reality (AR) environment and its high ecological validity enables conducting of novel behavioral paradigms, which place unique demands on the brain and elicit different networks to respond, in a very fine-grained manner. Such brain activation is crucial as it amplifies the subtle variations in brain functions, especially in small brain regions, and captures more behaviorally relevant information than resting-state functional connectivity. For example, eye tracking during pro-and anti-saccade paradigms (with real-time tracking of saccade parameters) is a non-invasive and sensitive way to assess 1) altered patterns of oculomotor behavior, and consequently, 2) subtle cognitive decline in subjects with MCI and patients with AD.

In addition to tracking saccade parameters, the real-time eye tracking utilizes AR headset's variable background luminance level and eye images captured at 60 Hz using the four infrared eye tracking cameras to precisely quantify the dynamic of task-evoked pupillary responses (TEPR) during non-luminance-mediated and luminance-mediated generation of reflexive and voluntary saccades. It is suggested that pupil size as a window on neural mechanisms underlying cognition and have identified the convergence of cognitively relevant signals onto the two brainstem nuclei: 1) the LC-NE neuromodulatory system, and 2) the SC, specifically the intermediate layers (FIG. 13). However, saccade parameters and pupil size alone are not enough and may be combined with simultaneous measurement of neural activity in multiple brain regions. That is why the proposed technological platform also allows task-evoked functional PET (fPET) imaging using hybrid bolus infusion injection of radiotracer.

4.1.1. Preliminary Experimental Results of heart Rate and Eye Tracking.

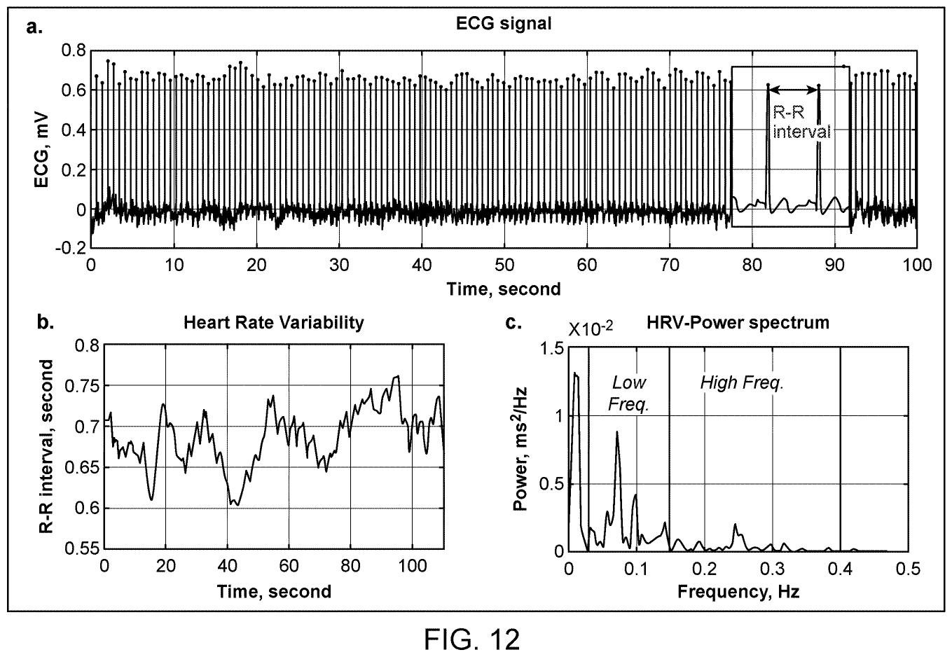

Heart rate and HRV are indicators of autonomic nervous system functions. Studies have found that the HRV provides an index of the parasympathetic nervous system, which is linked to a range of psychophysiological processes including cognitive functions, emotional regulation, and the management of stress. The ECG signal using a high-resolution 3-leads portable ECG sensor has been measured. The R-peaks in the ECG data were detected and the RR interval between each peak was calculated to generate the HRV curve (FIG. 12). Analysis of HRV was focused on both time-domain and frequency-domain measures. Time domain analysis includes metrics like the standard deviation of RR intervals (SDNN) and the percentage of consecutive RR intervals over 50 ms (pNN 50) were performed. The RR intervals were also transformed into the frequency domain, and its power spectrum may be segmented into three frequency bands: very low frequency (VLF; 0.0037-0.04 Hz), low-frequency band (LF; 0.04-0.15 Hz), and high-frequency band (HF, 0.15-0.4 HZ).

The energy ratio between LF and HF and other ECG parameters enabled evaluation of the focus and stress levels of the participants, which provided information on task difficulty, task engagement, and attention level from another dimension. The AR headset was also used to provide immersive visual and audio stimulations to the participants during the study. A customized program was developed to deliver visual tasks (pro-/anti-saccade and oddball) with the ability to change the frequency and duration of the tasks to control the difficulty level and cognitive load of the task. The eye movements were captured by two infrared eye cameras for each eye (FIG. 7). Eye rotation angle, pupil diameter, and 2D gaze coordinates were detected using the built-in program at a frequency of 60 Hz. The rest of the eye parameters including the pro-and anti-saccade speed, reaction time, amplitude (degree), direction error rate, and disengagement rate may be calculated from the gaze coordinates (FIG. 7).

4.1.3. Preliminary Experimental Results of fPET Using Anti-Saccade Task.

The pilot fPET study of saccade tasks using a clinical PET/MR scanner has shown promising results in distinguishing brain activation patterns between young and older participants in certain brain areas. For this study, n=17 younger (18-27 years, mean 21 years) and n=20 older (68-79 years, mean 74 years) participants with no current diagnosis of psychiatric illness were recruited. Participants were positioned supine in the scanner bore of a 3 Tesla Siemens Biograph mMR scanner. FDG was administered to the participant as a 50% bolus and 50% infusion. FDG was infused at a rate of 36 mL/hr for 50 mins. Arterial blood sampling was performed automatically for the first 3 min at a rate of 4 mL/min (Allogg) and manually at 5, 10, 20, 35, and 50 min.

Anti-saccade Task. The task was presented on an LCD screen via a mirror placed on the RF-coil. During task periods, participants alternated between short-duration blocks of anti-saccade, prosaccade, and null trials. Dynamic PET images were reconstructed and modeled by GLM following the technique.

### 4.1.4. Aim 1, Task 1: Development and Refinement of Immersive Behavioral Protocols.

The preliminary result may be used to further refine and optimize brain stimuli and tasks to test saccade prediction and pupil size measurement techniques using headsets with eye trackers. Measures of oculomotor function (including correct and incorrect anti-saccades and prosaccades, saccadic reaction time, anticipatory saccades, directional errors, saccade gain, and pupil size) may be included during anti-saccade, no-saccade, prosaccade and oddball tasks. Participants may receive instructions regarding the structure of the experiments, and they may be asked to practice the tasks before the experiment.

During the scan, they may complete two blocks of saccade tasks, followed by two blocks of oddball tasks. Each block may progressively increase in difficulty to impose varying cognitive load levels. The saccade task starts with a fixation cross displayed at the center of the virtual screen of the AR glass. The color of the cross cues the participants to execute a prosaccade (looking towards a target) or an anti-saccade (looking away from the target in the opposite direction). The sequence of saccade types and directions may be randomized.

Participants may be asked to hold their gaze in the position until the target disappears. Since a minimum of 90 ms is required for a visual signal to propagate through the oculomotor system and trigger a saccade, saccades occurring 90-800 ms post-stimulus were grouped into task-relevant responses, and those occurring below 90 ms were classified as anticipatory saccades. For the viable saccades, the reaction time, velocity, amplitude (degree), disengagement rate, and direction errors were further calculated.

Trials where participants looked away from the fixation point and did not return were marked as disengaged. Blink rate and blink durations were also recorded for analysis. In the oddball experiments, the participants may be presented with two visual stimuli: a blue rectangle as the standard stimulus, and a blue triangle as the target stimulus. In the first block of the oddball experiment, the stimuli may be displayed for 500 milliseconds with a 500-millisecond interval. For the second block, the stimuli may be displayed for 400 milliseconds with a 400-millisecond interval to increase the cognitive load of the experiments. Participants may be instructed to press a button upon the appearance of the target stimulus. Rest may be incorporated within and between task blocks. The order of stimuli may be randomized, with a consistent ratio of standard to target stimuli at 80% and 20%, respectively.

### 4.1.5 Aim 1, Task 2: Optimization of Fpet With Behavioral Tasks.

First, there may be an optimized approach to combining fPET with oculomotor behavioral tasks in n=5 healthy subjects, aged 18-50 years, screened for major medical and neurological conditions. Subjects may be asked to fast for 4 hours before the PET scan, and blood glucose levels may be confirmed to be in the normal range. First, the headset may be fitted to the participants and calibrate its eye-tracking cameras using the calibration software.

The start of data acquisition may be synchronized among (for both visual stimulation and eye tracking) ECG sensors, PET-CT scanner, and EMMT to within a few tens of milliseconds using the local time on the internet. All data may be recorded in list mode with time stamps and time markers. The participants may undergo a 70-minute functional 18F-FDG PET scan on a Siemens Biograph mCT PET-CT, wearing the and EMMT sensors for head motion tracking and compensation.

An initial bolus injection comprising 30% of the FDG dose (1.5 mCi) may be administrated intravenously at the beginning of the PET scan, and the remaining 70% of the dose (3.5 mCi) may be administered as constant infusion throughout the rest of the scan using an automatic infusion pump. Plasma radioactivity levels may be measured automatically for the first 3 min at a rate of 4 mL/min (Allogg) and manually at 5, 10, 20, 45, 60, and 70 min. Decay correction may be performed on all blood samples. The motion-corrected PET list-mode date may be histogrammed into time frames to form the dynamic dataset and may be reconstructed with both OSEM and kernel-based deep-learning methods to mitigate noise resulting from the utilization of short frame sizes (see Aim 2). The visual anti-saccade and oddball stimuli may be viewed through the headset.

MRI acquisition for brain co-registration and segmentation:

Subjects may undergo a standardized MRI on a 3T Siemens PRISMA system.

Volumetric 3D T 1 -weighted MPRAGE images may be used for within-subject anatomical localization and segmentation. Parameters: TR/TE=2400/2.96 ms, flip angle=9 degree; FOV=256 mm, matrix size=256 [symbol] 256, and 208 sagittal slices. A voxel size of 0.5 [symbol] 0.5 [symbol] 0.5 mm3 has been achieved using k-space interpolation in all three directions. Dynamic FDG-fPET images may be spatially aligned using FSL MCFLIRT. A mean FDG-PET image may be derived from the dynamic time series and rigidly normalized to the subject's 3D T 1 MPRAGE using ANTs. The dynamic FDG-fPET images may then be normalized to MNI space using the rigid transform in combination with the non-linear T1 to MNI warp. fPET images may be spatially smoothed using a Gaussian kernel of 12-mm FWHM. The mean of/PET signal across the entire gray matter mask may then be estimated and submitted for statistical analysis. Both general linear model (GLM) and independent component analysis (ICA) methods may be used to model task related brain activations.

### 4.1.6. Aim 1, Task 3: Within Subject Metabolic Connectivity for Brain Network Evaluation.

The motion-corrected PET image from the final 20 minutes may be registered to a template MR to generate the brain region masks. A GLM with three regressors may be employed to fit the time activity curves (TACs), yielding a beta-estimate of each regressor at each voxel. The averaged time course across all the gray matter voxels with a third-order polynomial to serve as the baseline regressor may be modeled. One regressor may be included for each task (anti-saccade and oddball), defined as a linear ramp function with a slope of 1 during task engagement and 0 otherwise. The parametric beta image of each task may undergo a Z-score transformation to generate the final activation image. The mean z-score may be extracted for each brain region.

Furthermore, TACs may be modeled using a two-compartment kinetic model with a time-variant k3 parameter to reflect the dynamic changes of k3 throughout the study (see Aim 2.). After grouping voxels into brain regions, the metabolic connectivity matrices of regional TACs and activation connectivity matrices of the dynamic k3 using Pearson's partial correlations may be computed. Additionally, the correlations between the brain regions and HRV, pupil size, and saccade parameters may also be calculated. Graph-based methods may be applied to evaluate interactions between brain regions and to identify functionally connected nodes for each cohort (HC, MCI, and AD). Since the TAC and dynamic k3 slopes vary due to task-related activations, a sliding window to select a subset of consecutive time points from the TAC and k3 curves may also be applied. The connectivity matrices of each time window may be computed. By analyzing the correlation coefficients across the windows, how the connectivity pattern fluctuates between regions in response to cognitive tasks can be assessed.

4.1.7. Aim 1, Task 4: Testing the Optimized fPET Protocol Synchronized to Cognitive Tasks in Vivo in 60 Subjects:

FDG-PET is an important tool to assess declining cerebral glucose metabolism in aging and Alzheimer's disease (AD) and is currently the clinical standard-of-care for differentiating AD from frontotemporal dementia in cognitively impaired individuals. N=30 subjects and n=30 subjects may be recruited. Note that the optimized behavioral protocols may be implemented on headset to perform behavioral fPET human subject studies in parallel with the studies.

### Inclusion Criteria

To evaluate the immersive behavioral fPET on a wide adult age range, 20 HC adults may be recruited, 10 of whom may be aged 30-60 years and 10 aged 60-90 years. To demonstrate the feasibility of the platform to study AD, 40 subjects (20 MCI+20 AD), aged 60-90 years, with a clinical symptom of MCI or early AD and evidence of B-amyloid plaque deposition in the brain, confirmed with a positive amyloid PET scan (40 centiloids) may also be recruited. Sex as a biological variable may be addressed, and enrolling approximately 50% female subjects is planned. Subjects may be recruited from the ongoing aging studies at BHII, in which currently more than 400 subjects are enrolled, and typically approximately 20-30 subjects are screened per month.

### Exclusion Criteria

All subjects enrolled in studies at the BHII undergo a comprehensive clinical, laboratory, and cognitive evaluation following National Alzheimer's Coordinating Center guidelines, which includes standardized medical history and physical examination, neurological and psychiatric examination, neuropsychological assessments, baseline blood tests, as well as structured interviews with informants. Exclusion criteria include 1) neurocognitive or neurodegenerative disorders, such as AD, frontotemporal dementia, normal pressure hydrocephalus, Lewy body dementia, Parkinson's disease, 2) other neurological and psychiatric disorders, such as brain tumors and infarctions, major depression and schizophrenia, and 3) pregnancy or contraindication to MRI, due to metal, claustrophobia, or weight limits.

fPET and MR Image Acquisition and Quantification:

All 40 subjects may undergo the optimized fPET protocol and volumetric MRI. All subjects may undergo the behavioral tasks using the AR headsets (one headset may be and the second headset may be shipped). Dynamic fPET images may be quantified and analyzed using GLM and ICA approaches. Analyses may be performed comparing task-based fPET signal in the younger (30-60 y) versus older (60-90 y) healthy adults, as well as healthy versus cognitively impaired older adults. Subgroup analyses may also be performed, stratified by sex/gender, to explore whether fPET signal is modified by sex or gender.

### Expected Outcomes for Aim 1:

Successful completion of this aim may demonstrate the feasibility of the fPET protocol, when combined with oculomotor behavioral tasks (i.e., anti-saccade and oddball paradigms. Differences in fPET signal are anticipated between the normal aging groups and between the normal versus cognitively impaired groups using oculomotor tasks. Specifically, less of an increase in fPET signal in memory regions of the brain in the cognitively impaired group compared to the healthy older group, with possible areas of increased and decreased fPET signal in other regions of the brain as compensation is anticipated. Differential patterns of increased and decreased fPET signal in comparing the younger versus older cognitively healthy groups may also be explored. Successful completion of this aim may provide the expected patterns of fPET signal related to normal aging versus AD, as expected using the new upright Prism-PET platform but at much higher spatial resolution and SNR.

### Alternative Path for Aim 1:

Adequate signal change may not be received on fPET across all regions in the oculomotor circuit. This issue may be mitigated by 1) optimizing the ON-OFF period of each stimuli (e.g., duration and number of anti-saccades/prosaccades in each test session, number of test sessions, and duration of resting state between sessions), and 2) improving the signal-to-noise ratio (SNR) in the data-driven dynamic image reconstruction (see Aim 2). This issue may also be fundamentally addressed by utilizing the ultra-high resolution and high sensitivity Prism-PET scanner in Aim 3 which may enable higher SNR specially in small brain nuclei (such as SC and LC). In addition, there may not be adequate power to detect statistically significant differences using JUST/PET signal among the 3 groups, especially due to heterogeneity in AD. However, multimodal quantification of oculomotor behavior (i.e., saccade parameters, dynamics of pupil size, and HRV) may be combined with dynamic fPET data to precisely and reproducibly probe functional connectivity pathways in HC, MCI, and AD while reducing effects of sample heterogeneity.

### 4.2. Aim 2: Development and Testing of the Ultra-High Resolution and Upright Prism-PET/EMMT Brain Scanner.

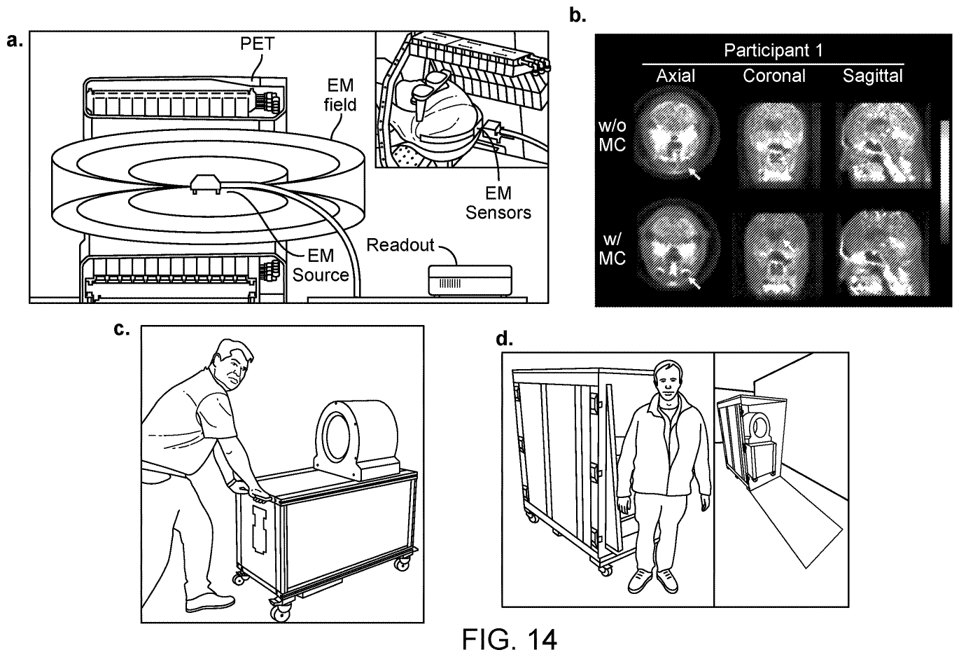

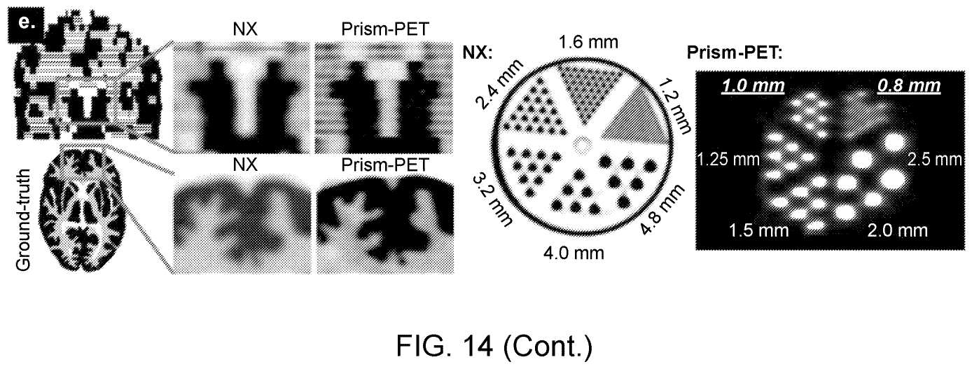

To fully exploit the benefits of fPET and mitigate its limitations (i.e., increase fPET sensitivity by reducing frame time interval; eliminate motion artifacts and use high-resolution PET imaging to enable analysis of brain activation in small regions), an ultra-high sensitivity, ultra-high resolution, motion-compensated, portable, and upright Prism-PET brain scanner is proposed (a recently built prototype is shown in FIG. 14). What enables imaging in the sitting position using immersive behavioral tasks is the recently developed wearable electromagnetic motion tracker (EMMT) which is capable of achieving sub-millimeter motion tracking accuracy for both small (mm-scale) and large (cm-scale) head movements.

Preliminary experimental results of the constructed portable and ultra-high-resolution Prism-PET scanner in the supine position. FIG. 14a shows the schematic of a recently developed Prism-PET scanner with EMMT. EMMT comprises a source, an array of sensors, and a readout electronic unit (REU). The REU digitizes signals from each sensor and captures precise rotational and translational movements in real time. Tracked motion in the EMMT coordinate system is synchronized with the PET list-mode data and transformed into the scanner coordinate system by locating paired positions in both systems. The optimal rigid motion is estimated using singular value decomposition (SVD). One human participant was enrolled in an institutional review board-approved study, for evaluation of the proposed EMMT and event-by-event motion correction in clinical brain PET imaging. FIG. 14b shows the reconstructed MK6240 PET images with (w/) and without (w/o) head motion correction (MC).

The images highlighted two structures, namely the eyeball and ventricle, with a substantial reduction in motion-induced blurring, as indicated by white arrows. FIG. 14c shows the recently constructed portable cart and scanner cover for the proposed Prism-PET scanner.

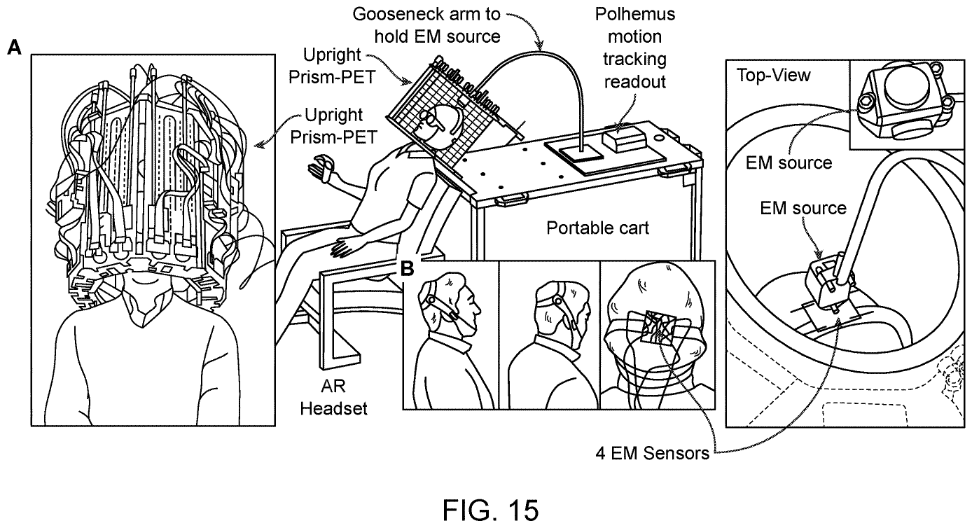

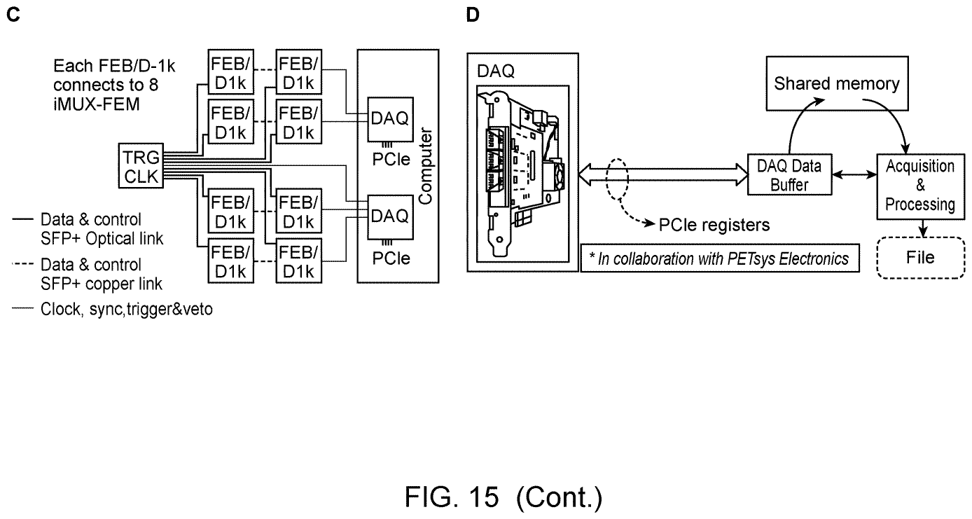

The gantry (which is comprised of cold plates) is also already constructed and can accommodate 12 axial rings of Prism-PET detector modules. The funding requested in this proposal may allow the applicants to 1) finish the development of this scanner using the recently developed interleaved multiplexing (iMUX) readout and extend its axial field-of-view (AFOV) from 1 ring (i.e., 2.5 cm) to 12 rings (i.e., 33 cm), 2) integrate it with EMMT, and 3) convert the supine configuration to the upright sitting position. FIG. 14d shows the portable crate made to house the scanner for moving or shipping it to another facility or institution. FIG. 15e shows experimental results of phantoms. The NeuroExplorer (NX) can resolve 1.6 mm or larger regions and thus, cannot resolve the 0.8-mm white matter layers axially. However, Prism-PET images demonstrate accurate delineation of both gray matter, white matter, and non-activity ventricle regions in all views. Most importantly, all 0.8-mm white matter layers can be perfectly resolved, demonstrating ultra-high-resolution performance in all directions.

### 4.2. 1. Aim 2, Task 1: Construction of Upright Prism-PET/EMMT Scanner.

A new portable cart with an upright gantry may be developed to allow PET imaging in an upright sitting position (FIG. 15A). This may also improve comfort and compliance among subjects with back pain or claustrophobia. Most importantly, what enables imaging in the sitting position with free head motion is the recently developed EMMT with wearable EM sensors that can compensate for motion in real time (FIG. 15B). Note that for the full-scale development of ultra-high resolution PET scanner for human brain imaging, reading individual channels is not practical and pixel-to-readout multiplexing may be incorporated to reduce complexity, cost, power consumption, and heat output.

iMUX boards have been developed and achieved perfect signal demultiplexing without artifacts (see published results from iMUX). For the full-scale development of the human brain scanner with 33-cm axial FOV, 12 axial rings of 4-to-1 coupled Prism-PET detector modules may be fabricated and characterized, with each ring comprising of 40 detector modules. These modules may be assembled onto each cold plate to form the super-modules and then mount the super-modules onto the gantry. For the construction of the EMMT system, a boom-stand (or a gooseneck arm) may be built so the electromagnetic (EM) source can be placed in close proximity to the top of the head. A wearable EM sensor head-strap may be worn by the human subjects to position the sensor array at the top of the head.

This full-scale Prism-PET/EMMT scanner (i.e., 12 axial rings of detectors) with 480 detector modules may connect to 120 ASICs (or 60 iMUX-FEM boards with each housing 2 ASICs), and the gamma events detected by these ASICs may be readout using 8 TOF Front-End Board D (FEB/D). FEB/D collects event records from the ASICs and sends these to the two data acquisition (DAQ) modules in the DAQ computer (FIG. 15C). It provides power, configuration signals, and clock and synchronization signals to the ASICs, and adjustable bias voltages for the SiPM arrays. Together with the clock-and-trigger (CLK-TRG) module it allows system wide coincidence detection in the firmware. During the scanner commissioning phase, new firmware and software solutions may be developed for transmission and processing of coincidence events, data reduction schemes using energy selection, and data reduction schemes using online software data processing (FIG. 15D).

This may be used because a large data rate is expected to be generated by the readout system, where each Gamma interaction in the crystal generates several digitized events, one for each channel where the measured charge crossed an acceptance threshold. Once the scanner development is complete, the chiller may be connected to the gantry cold plates, perform Prism-PET scanner calibration and normalization, perform EMMT system calibration, align Prism-PET and EMMT coordinate systems, and evaluate the system's temperature stability. The same normalization procedure developed successfully may be used for normalizing the previously built proof of-concept Prism-PET brain scanner. Depth-encoded crystal look-up tables may be created for all detector modules from flood histograms.

4.2.2. Aim 2, Task 2: Image Reconstruction and Noise Reduction for High-Temporal-Resolution fPET.

The patient's motion may be measured in real-time using multiple calibrated EMMT sensors and generate synchronized EMMT list-mode data (EMMT-LMD) and PET list-mode data (PET-LMD). To achieve high temporal resolution in fPET, MC-LMD data may be divided into short temporal frames. However, short-time frames contain fewer counts in each frame and hence increase noise in reconstructed images. To reduce image noise, dynamic PET data may be reconstructed by kernel-based methods, which have been used to reconstruct dynamic PET image at 0.1 s temporal resolution. Specifically, the original kernel method divides the dynamic PET data into a small number of frames and reconstruct these frames into composite images.

Features are identified from the composite images for each pixel and then used to construct a kernel matrix for representing the spatial distribution of the tracer at each time point. The kernel method can substantially reduce noise in reconstructed dynamic PET images, especially those of short frame durations. The kernel method can easily incorporate anatomical information from CT or MR as well as perform direct parametric reconstruction for the Patlak model and two-tissue-compartment model.

### Milestones.

Completion of immersive AR-based behavioral anti-saccade and oddball tasks with real-time eye and heart-rate tracking. The optimized anti-saccade and oddball tasks may be implemented and real-time tracking of saccade parameters and pupil size in the AR headsets may be provided.

### Quantifiable Criteria for Success:

Completion of oculomotor-/PET validation. The oculomotor-fPET may be validated using human subjects and compare against the original preliminary data (see FIG. 14d).

### Quantifiable Criteria for Success:

Due to higher ecological validity of the immersive AR environment and more precise eye tracking, enhanced brain activation, task engagement and task compliance are expected.

Completion of oculomotor brain mapping using multimodal task-evoked recording. The optimized fPET protocol synchronized to behavioral oculomotor tasks in vivo in 60 subjects may be validated.

### Quantifiable Criteria for Success:

BIDS-compliant fPET cognitive brain data of all 60 subjects may be shared (together with measured subjects' saccade parameters and pupil size dynamics) on the OpenNeuro platform or BRAIN initiative repository. It is expected that the oculomotor behavior and functions are organized across the entire brain regions (even including small brainstem nuclei such as SC and LC) in healthy and pathological conditions (i.e., MCI and AD).

### Quantifiable Criteria for Success:

After completion, the scanner may be powered on and calibrated using the procedure developed by PETsys. The calibration files may quantify the test result for each multiplexed channel and dead channels, or loose connections can be identified and repaired. The motion-compensated imaging performance of the scanner may be tested using ultra-micro hot-spot and 3D Hoffman brain phantoms filled with 18F activity.

4.3. Aim 3, (n=45): Immersive task-Evoked SPH Tracking with Simultaneous Upright fPET to Study the Link Between Oculomotor Behavior and Neural Circuitry of Cognition.

High temporal and spatial resolution MIf-PET (FIG. 1) may provide an unprecedented opportunity to quantify brain activation that can be potentially superior to functional magnetic resonance imaging (fMRI) because it's a fully quantitative index of neuronal activity and can be utilized in a more naturalistic setting by imaging subjects in an upright sitting position. Upright MIf-PET may provide 1) multimodal sensory stimulation using immersive visual information, auditory information, and haptic feedback; and 2) multimodal recording using head and eye tracking in addition to brain mapping using high temporal and spatial resolution fPET. The integration and synchronization of these tools may help study changes in brain functional connectivity with a precision not attainable ever before and may provide invaluable insights into these unique behavioral and cognitive processes in normal and eventually those with incipient brain disease.

4.3.1. Aim 3, Task 1: Clinical translation of MIf-PET.

Study design and patient enrollment. n=15 HC and n=15 AD subjects may be recruited over years 4 and 51, aged 60-90 years (AD again confirmed by positive amyloid PET). The upright Prism-PET/EMMT scanner may be placed inside the portable shipping crate (see FIG. 14d) and recruit n=15 MCI subjects may be recruited for the clinical translation of MIf-PET. The goal may be to recruit 50% male/female.

Upright Prism-PET/EMMT image acquisition and analyses: All 45 subjects may undergo the optimized behavioral and fPET protocol, as noted in Aim 1, on the newly developed upright Prism-PET/EMMT scanner, which may provide a more naturalistic environment for subject assessment and also provide high-resolution to study smaller brain structures that are currently not quantifiable on conventional PET. Subjects may also perform the oculomotor behavioral tasks using the newly developed AR goggle technology.

MR image acquisition: Subjects may undergo 3D T 1 MPRAGE on a 3T Siemens PRISMA scanner for co-registration and segmentation. Analyses may again be repeated within subgroups, stratified by sex/gender.

### Expected Outcomes and Impact of Aim 3:

It is expected that the upright, motion-compensated Prism-PET scanner may allow high spatial resolution across the entire FOV, even in the presence of free head motion, due to both high-resolution DOI localization (<2 mm FWHM) and high-resolution motion tracking (<0.5 mm). Combined with the high temporal resolution of fPET, it is expected that there may be differences in regional fPET signal when comparing cognitively healthy vs impaired older adults, particularly in the memory regions of the brain.

### Alternative Path for Aim 3:

Statistical power may again be limited. However, the multimodal quantification of oculomotor behavior (i.e., saccade parameters, dynamics of pupil size, and HRV) may be combined with dynamic PET data to precisely and reproducibly probe functional connectivity pathways in HC, MCI, and AD while reducing effects of sample heterogeneity.

### C. Systems and Methods for Detecting Conditions in Subjects Provided with Multimodal Stimuli Using Multimodal Recordings

Functional imaging may use functional magnetic resonance imaging (fMRI) to detect blood oxygen level dependent (BOLD) contrast. However, the clinical MRI scans may be extremely noisy and involve the participants to maintain a supine position, both of which have been shown to affect brain functions and introduce bias into the results. Furthermore, fMRI may provide a limited, indirect measure of brain activity through blood oxygen level and may lack the quantitative capacity to study the precise levels of brain activity.

In contrast to fMRI, functional Positron Emission Tomography (fPET) may offer inherent advantages. First, fPET can image a wide range of specific targets such as neurotransmitters, neuroreceptors, protein analogs, cellular-level glucose consumption, cerebrospinal fluid, and more. Second, PET's operation may be quiet, minimizing the interference with brain functions, especially within brain's auditory processing network. Third, fPET may provide direct and quantitative assessments that links to the distribution of the specific biomarkers.

While use of PET may resolve some of the issues with MRI, clinically available PET scanners may suffer from limited sensitivity and poor spatial resolution. The images may be harmed by partial volume effects, and none of the important small brain structures and nuclei can be accurately measured using fPET. Additionally, the fPET protocol may involve head constrains (to reduce the motion artifacts) and participants interacting with the tasks or stimulus through a small mirror attached to the bore of the scanner. The attachment of the mirror may reduce participant engagement level and task performance.

The human brain may be a complex mechanism, interfacing with the environment via multi-dimensional inputs including the sight, touch, hear, and smell, among other senses. The brain also may regulate the activity of other organs to meet behavior and cognitive demands (e.g., modulating heart rate and breathing rate in response to stress). Given this complexity, measuring the brain activation itself may be insufficient to thoroughly understand and model the cognitive functions, or to identify anomalous or alerted networks associated with brain disorders such as neurodegenerative diseases.

Referring now to FIG. 16, depicted is a block diagram of a process for measuring brain functionality using a multi-dimensional, motion-tracked, immersive function PET (MIf-PET) platform. The platform may have a number of advantages. First, the platform may include an ultra-high performance upright Prism-PET brain scanner. The Prism-PET brain scanner may achieve high spatial resolution and have high detection sensitivity, allowing the imaging of small brain nuclei. The subject can be imaged in an upright posture within the PET brain scanner, further reducing the bias caused by the body position and reduce the claustrophobia. Moreover, the scanner may cover the head of the participants and leaving the rest of body to move free and enable cognitive or behavior tasks involving large leg or arm movements or interactions.

Second, the platform may be portable. The system may be mobile and portable which could be disseminating to other institutions for multi-center studies or local brain disorder scanning. A cart may be made to house the scanner for moving or shipping. Third, the platform may allow for sub-millimeter motion tracking and correction using a motion tracker in the PET brain scanner. The electromagnetic motion tracker (EMMT) can perform sub-mm motion tracking and correction, eliminating the inclusion for head restraints during the scans and the participants can freely interact with the augmented reality tasks.

Fourth, the platform may include immersive augmented reality (AR) glasses for visual stimulus. The AR glass may be synchronized with the systems and a software has been customized to perform immersive visual stimulus (e.g., saccade and anti-saccade tasks, oddball tasks) to the subject. This way, an enhanced task engagement may be achieved and the target brain functions may be more effectively stimulated, surpassing the other approaches and protocols. Fifth, the platform may include an eye movement tracking. The high-resolution infrared eye camera of the AR glasses may provide accurate gaze and pupil tracking. With the customized algorithms, eye movement features which are governed by a range of critical brain structures (e.g., locus Coeruleus, Dorsal Raphe, Superior Colliculus, Basal Ganglia, and Frontal Cortex) can be extracted, providing another dimension of information necessary for modeling brain functions.

Sixth, the platform may allow for electrocardiogrameasurements. Heart rate may include indicators of autonomic nervous system functions. the heart rate variability (HRV) may provide an index of the parasympathetic nervous system, linked to a range of psychophysiological processes including cognitive functions, emotional regulation, and the management of stress. The ECG sensor may be synchronized with the PET scanner for the tracking of heart rate features (e.g., by providing a clock signal to all sensors). In addition, the platform may provide for extendibility. Other peripheral physiological measurements can be easily included into the platform. For example, the sensors can include respiratory, electroencephalogram (EEG), electrodermal activity (EDA), and electromyography (EMG), among others, into the platform around the fPET scanner.

Referring now to FIG. 17, depicted is a block diagram of a system 100 for detecting conditions in subjects provided with stimuli. The system 100 may include at least one data processing system 105, at least one testing environment 110, and at least one display 115, communicatively coupled with at least one network 120. The data processing system 105 may include at least one test handler 125, at least one image retriever 130, at least one signal analyzer 135, at least one model trainer 140, at least one model applier 145, at least output handler 150, at least one data analysis model 155, and at least one database 160, among others. The testing environment 110 may include at least one headset 165, at least one tomography scanner 170, at least one motion tracker 175, one or more sensors 180A-N (hereinafter generally referred to as sensors 180), and one or more input/output (I/O) devices 185A-N (hereinafter generally referred to I/O devices 185), among others. At least one subject 190 may be situated within the testing environment 110. Each of the components in the system 100 (excluding the subject 190) as detailed herein may be implemented using hardware (e.g., one or more processors coupled with memory), or a combination of hardware and software as detailed herein in Section D. Each of the components in the system 100 may implement or execute the functionalities detailed herein, such as those described in Sections A and B.

In further detail, the data processing system 105 may (sometimes herein generally referred to as a computing system or a server) be any computing device comprising one or more processors coupled with memory and software and capable of performing the various processes and tasks described herein. The data processing system 105 may be in communication with the testing environment 110 (including the tomography scanner 170, the headset 165, the motion tracker 175, the sensors 180, and I/O devices 185) and the display 115, via the network 120. The data processing system 105 may provide a common clock signal to the components in the testing environment 110 (including the tomography scanner 170, the headset 165, the motion tracker 175, the sensors 180, and I/O devices 185) to facilitate synchronization of data. The data processing system 105 may be situated, located, or otherwise associated with at least one server group. The server group may correspond to a data center, a branch office, or a site at which one or more servers corresponding to the data processing system 105 is situated.

Within the data processing system 105, the test handler 125 may send instructions to provide stimuli to the subject 190 via the headset 170 or the I/O devices 185. The image retriever 130 may receive data defining tomograms of a head of the subject 190 via the tomography scanner 170. The signal acquirer 135 may obtain a response signal associated with the subject 190 from the one or more sensors 180 in response to the provision of the stimuli. The model trainer 140 may initialize and train the data analysis model 155 to determine conditions in a nervous system of the subject using training data. The model applier 145 may apply the data analysis model 155 to the tomograms and response signal to determine a condition in a nervous system of the subject 190. The output handler 150 may generate an output for presentation based on the condition determined for the subject 190.

The data analysis model 155 may include a machine learning model or a statistical model, among others. The machine learning may be any architecture to identify or determine a condition of a nervous system in the subject 190. The machine learning model for the data analysis model 155 may include, for example, an artificial neural network (ANN) (e.g., a deep learning architecture including feedforward neural network, convolutional neural network, a recurrent neural network, a generative adversarial network, or a transformer model), a decision tree, or an ensemble model, among others, or any combination thereof. The statistical model for the data analysis model 155 may include, for example, a clustering algorithm (e.g., k-means clustering), a support vector machine (SVM), a random forest model, a Bayesian classifier, a principal component analysis (PCA), or a regression model (e.g., a logistic or linear regression model), or any combination thereof (including machine learning architecture). In general, the data analysis model 155 may include one or more inputs and one or more outputs. The inputs and outputs of the data analysis model 155 may be related to one another via a set of weights in accordance with the architecture. The inputs may include the tomograms and the corresponding response signal from the subject 190. The output may include the condition associated with the nervous system of the subject 190.

The testing environment 110 may correspond to or include a defined space, region or volume in which the subject 190 is under evaluation for one or more conditions in the nervous system. The headset 165, the tomography scanner 170, the motion tracker 175, the one or more sensors 180, and the I/O devices 185, among others are arranged, disposed, or situated within the same space as the subject 190. The volume forming the testing environment 110 may separate the components and the subject 190 from a volume outside the testing environment 110. For example, the testing environment 110 may be a room in a clinic, hospital, or test site in which the components (e.g., the headset 165, the tomography scanner 170, the motion tracker 175, the sensors 180, and the I/O devices 180) reside, along with the subject 190. A clinician or a technician may be present with the subject 190 in the testing environment 110.

Within the testing environment 110, the subject 190 may be at risk of or under examination for one or more conditions. The subject 190 may include, for example, a human or an animal, among others. The condition to be determined may be associated with a nervous system of the subject 190. The nervous system may include, for example, a central nervous system (CNS) (e.g., a brain and a spinal cord) and a peripheral nervous system (PNS) corresponding to nervous outside the CNS, including a somatic nervous system (e.g., muscle, skin, and sensory organs) and an autonomics nervous system (ANS) (e.g., heart, respiration or glandular secretion), among others. The condition may include, for example, a tumor, brain injury, epilepsy, neurodegenerative disease (e.g., Alzheimer or Huntington's disease), stroke (e.g., ischemic or hemorrhagic), a biomarker (e.g., related to disease or disease), disease response, drug response, movement disorder (e.g., Parkinson's, tremor, dystonia), or behavioral condition, among others.

The headset 165 may be a wearable computing device comprising one or more processors coupled with memory and software and capable of performing the various processes and tasks described herein. The headset 165 may be in communication with the data processing system 105, the display 115, the tomography scanner 170, the motion tracker 175, the sensors 180, and I/O devices 185, among others, via the network 120. The headset 165 may provide virtual reality (VR) or augmented reality (AR) functionalities to the subject 190. The headset 165 may be fitted, arranged, or otherwise situated on a head on the subject 190. The headset 165 may include at least one display and at least one loudspeaker to present audiovisual stimuli to the subject 190. The display may be generally situated facing one or both eyes of the subject 190 to provide visual stimuli. Each loudspeaker may be generally situated about a corresponding ear of the subject 190. The headset 165 may, for example, be the AR glasses detailed herein in Sections A and B.

The tomography scanner 170 (sometimes herein generally referred to as an imaging device or an image acquirer) may be any device for acquiring tomograms of the subject 190 (e.g., the head including the brain). The tomography scanner 170 may be in communication with the data processing system 105, the display 115, the headset 165, the motion tracker 175, the sensors 180, and I/O devices 185, among others, via the network 120. The tomography scanner 170 may scan a volume corresponding to the head of the subject 190 to acquire the tomograms in accordance with positron emission transmission (PET) or single photon emission computed tomography (SPECT) scanning technique. The tomography scanner 170 may generate a set of tomograms at a sampling rate (e.g., ranging between 15-500 Hz) over a time period.

The tomography scanner 170 may be arranged, positioned, or otherwise situated on a mobile structure (e.g., a cart, basket, suitcase, or vehicle). The subject 190 may remain in at least a partially upright posture within the volume scanning of the tomography scanner 170, while the tomograms are acquired by the tomography scanner 170. The tomography scanner 170 may omit or lack a subject table in which the subject 190 is at rest in a prone or supine position. The tomography scanner 170 may be the prism-PET scanner as detailed herein in Sections A and B. While primarily described in terms of PET or SPECT imaging, the tomography scanner 170 may use other imaging modalities, such as a magnetic resonance imaging (MRI) scanner, a nuclear magnetic resonance (NMR) scanner, X-ray computed tomography (CT) scanner, an ultrasound imaging scanner, or a photoacoustic spectroscopy scanner, among others.

The motion tracker 175 may be a wearable computing device comprising one or more processors coupled with memory and software and capable of measuring a motion of the head of the subject 190 within the tomography scanner 170. The motion tracker 175 may be in communication with the data processing system 105, the display 115, the headset 165, the tomography scanner 170, the sensors 180, and I/O devices 185, among others, via the network 120. The motion tracker 175 may be disposed, arranged, or otherwise situated within the scanning volume of the tomography scanner 170. In some embodiments, the motion tracker 175 may be disposed, arranged, or otherwise situated on the headset 165. The motion tracker 175 may determine measurements of a position, orientation, or movement of the head of the subject 190 relative to an electromagnetic source within the scanning volume of the tomography scanner 170. For instance, the motion tracker 175 may be the electromagnetic motion tracker (EMMT) as detailed herein in Sections A and B.

The sensors 180 may be any device for acquiring various signals associated with the subject 190. In general, the sensors 180 may acquire signals measuring physiological functions of the subject 190. The physiological functions may include, for example, a respiration, heart, a skin conductance, perspiration, eyes, muscles, or nervous system, among others. The sensors 180 may be in communication with the data processing system 105, the display 115, the headset 165, the tomography scanner 170, the motion tracker 175, and I/O devices 185, among others, via the network 120. The sensors 180 may include, for example, an eye tracker, an electrocardiogram (ECG) sensor, a respiration sensor, an electro-encephalogram (EEG) sensor, an electrodermal activity (EDA) sensor, or an electromyography (EMG) sensor, among others.

The sensors 180 may include any of the types of sensors or measurement devices detailed herein in conjunction with Sections A and B.

The I/O devices 185 may be any device to accept inputs corresponding to user interactions by the subject 190 and provide outputs (e.g., in the form of visual, audio, or haptic output) to the subject 190. The I/O devices 185 may be in communication with the data processing system 105, the display 115, the headset 165, the tomography scanner 170, the motion tracker 175, the sensors 180, among others, via the network 120. The I/O device 185 may include, for example, a keyboard, touch pad, touch screen, mouse or other pointing device, scroll wheel, click wheel, dial, button, switch, keypad, microphone, joystick, display, speaker, headphones, touchscreen, or a haptic controller, among others. In some embodiments, the I/O device 185 may be part of the headset 165. For instance, the I/O devices 185 may be communicatively coupled (e.g., via wired connection) with the headset 165 to facilitate VR or AR functionalities provided through the headset 165.

The display 115 may be communicatively coupled with the data processing system 105 or any other computing device comprising one or more processors coupled with memory and software and capable of performing the various processes and tasks described herein. The display 115 may be in communication with the data processing system 105, the headset 165, the tomography scanner 170, the motion tracker 175, the sensors 180, and I/O devices 185, among others, via the network 120. The display 115 may display, render, or otherwise present any information provided by the data processing system 105. In some embodiments, the display 115 may be situated within the testing environment 110. In some embodiments, the display 115 may be outside the testing environment 110.

Referring now to FIG. 18, among others, depicted is a block diagram of a process 200 of communicating with test environments in the system for detecting conditions in subjects provided with stimuli. The process 200 may include or correspond to operations performed in the system 100 to acquire tomograms and response data about subjects and determine conditions in nervous systems of subjects based on the acquired tomograms and response data. For the process 200, the subject 190 may be positioned, located, or otherwise situated in the testing environment 110. For instance, the subject 190 may be guided by a clinician or a technician into the room corresponding to the testing environment 110. The subject 190 may have a head 202 and a body 204. The head 202 may correspond to a portion of subject 190 above a neck region or shoulder. The body 204 may correspond to a remaining portion of the subject 190, beneath the neck region or shoulder. To undergo evaluation, the headset 165 may be secured, fitted, or otherwise situated on the head 202 of the subject 190. For example, the headset 165 including the motion tracker 175 may be strapped around the head 202 of the subject 190. In some embodiments, the motion tracker 175 may be secured, arranged, or otherwise situated on the headset 165 or the head 202 of the subject 190.

In addition, the head 202 of the subject 190 may be placed, moved, or otherwise disposed into the tomography scanner 170. The tomography scanner 170 may be situated, disposed, or arranged about at least the head 202 of the subject 190. The tomography scanner 170 may have at least one body structure 206 (sometimes referred herein as a gantry or a frame). The body structure 206 may support, contain, or otherwise include a set of detectors to accept, obtain, or otherwise acquire photons for imaging. For example, the set of detectors may be for PET or SPECT imaging to acquire photon emissions from the subject 190 position within the tomography scanner 170. Each detector may include a scintillation crystal coupled with a light sensor such as a photomultiplier, among others. The body structure 206 of the tomography scanner 170 may also define or include at least one scanning volume 208 (sometimes herein referred to as a port or opening). The scanning volume 208 may correspond to an aperture or a hollow opening in which at least the head 202 of the subject 190 is to be situated. The scanning volume 208 may have a height ranging between 20-50 cm and a width, length, or diameter ranging between 55-80 cm. The tomography scanner 170 may allow or permit the head 202 of the subject 190 with a degree of freedom within the scanning volume 208. The degree of freedom may correspond to a length or angle at which the head 202 is free to move or tilt within the scanning volume 208 defined by the body structure 206.

The subject 190 may be in an upright posture (sometimes referred herein as an upright position or an orientation) within the scanning volume 208 defined by the body structure 206 of the tomography scanner 170. The upright posture may correspond to at least a torso of the body 204 in an erect or vertical orientation. For instance, the subject 190 in the upright posture may be in a standing position (e.g., supported by feet on the ground of the testing environment 110) or in a sitting position (e.g., supported by a buttocks of the body 204 on a seat). A vertical axis formed by the torso (e.g., a spine or back) of the body 204 may be 60-90° relative to a ground of the testing environment 110 or the seat in which the subject 190 is positioned. In addition, the tomography scanner 170 may be arranged, angled, or otherwise oriented in accordance with the orientation of the body 204 of the subject 190. A vertical axis defined through the tomography scanner 170 may also correspond to the orientation of the head 202 or the torso of the body 204 of the subject 190. For example, the mobile structure carrying the tomography scanner 170 may be moved toward the subject 190, and the tomography scanner 170 may be positioned to be arranged about the head 202 of the subject 190 at an angle substantially equivalent (e.g., 80-95%) to the angle at which the head 202 or body 204 is in.

With the subject 190 equipped with the headset 165 situated in the tomography scanner 170, the subject 190 may be injected or administered with at least one radiotracer. The radiotracer may be a radioactive compound used to visualize chemical reactions within the head 202 or the body 204 of the subject 190. The radiotracer may include, for example, fluorodeoxyglucose (FDG) (e.g., 18F-FDG), Carbon-11 Methionine, F-18 Fluciclovine, Florbetaben, Fluorodopa (F-DOPA), C-Pittsburgh Compound-B (PiB), and Ga-68 DOTATATE, among others. The radiotracer may be administered (e.g., via a needle injection) to the subject 190 via a bolus (e.g., a concentrated dose) over a set amount of time (e.g., 15 seconds to 5 minutes) or via an infusion over a prolonged period of time (e.g., greater than 5 minutes to an hour), or a combination of bolus-infusion (e.g., a hybrid injection as detailed herein in Sections A and B). With the administration of the radiotracer to the subject 190, the data processing system 105 may be invoked (e.g., via a user interaction or detection of the administration of the radiotracer) to initiate functionalities as detailed herein.

The test handler 125 executing on the data processing system 105 may maintain a set of instructions 215A-N (hereinafter generally referred to as instructions 215). Each of the instructions 215 may be stored and maintained on the database 160 using one or more files.

Each instruction 215 may specify, identify, or otherwise define presentation of a stimuli 220 to direct the subject 190 to perform at least one task. The instruction 215 may define the virtual reality (VR) or augmented reality (AR) experience to be presented to the subject 190 via the headset 165. The stimuli 220 may include audio stimuli, visual stimuli, or haptic stimuli, among others, or any combination thereof. The instruction 215 may define or identify at least a portion (To) of a time period (T) over which to present the stimuli 220 to direct the subject 190 to perform the task. The instruction 215 may define or identify a remaining portion of the time period (T) over which to direct the subject 190 to rest. The time periods including the active portion and resting portion may be repeated any number of times.

Continuing on, the task provided via the stimuli 220 may activate at least a portion (e.g., a processing region) of the nervous system in the subject 190. The task may include, for example, a visual task (e.g., pro-saccade visual task, an anti-saccade visual task, or an oddball task), a behavioral task (e.g., a go/no-go task, exposure therapy, or flooding), a cognitive task (e.g., pattern recognition, cognitive therapy, a spatial reasoning test, or matrix reasoning test), or a videogame (e.g., an inflight simulator, a first-person shooter, or virtual board game), among others. The task may be performed by the subject 190 (e.g., via eye movement or via interactions with the I/O devices 185). The test handler 125 may provide, transmit, or otherwise send at least one instruction 215 to the headset 165. From the database 160, the test handler 125 may identify or select the instruction 215 from the set of instructions 215 on the database 160.

The selection may be received via input from the subject 190 or the clinician or technician examining the subject 190. In some embodiments, the test handler 125 may omit the sending of the instructions 215 to the headset 165.

With receipt from the data processing system 105, the headset 165 may execute in accordance with the instruction 215. The headset 165 may provide or present the stimuli 220 to direct the subject 190 to carry out or perform the task over the portion (To) of the time period (T). The headset 165 may also direct the subject 190 to withdraw or rest from the task over the remaining portion of the time period (T). For example, the headset 165 may render the visual stimuli via a display to the eyes of the subject 190 and play the audio stimuli via a loudspeaker to the ears of the subject 190. In some embodiments, the headset 165 may present audiovisual stimuli via the display or the loudspeaker (or both) to direct the subject 190 to perform the task (e.g., the visual task, behavioral task, cognitive task, or videogame via the I/O devices 185). The subject 190 in turn may interact using the I/O devices 185 in response to the presentation of the stimuli 220. In addition, the tomography scanner 170 may acquire a scan (e.g., a PET or SPECT scan) of the nervous system of the subject 190. The motion tracker 175 may measure a movement, orientation, and position of the head 202 of the subject 190. The sensors 180 may monitor for signals associated with various physiological functions of the subject 190. The I/O devices 185 may receive the interactions by the subject 190 in response to the provision of the stimuli 220.

In conjunction, the image retriever 130 executing on the data processing system 105 may retrieve, identify, or otherwise receive at least one scanning dataset 225 from the tomography scanner 170. The receipt of the scanning dataset 225 may be subsequent to the administration of the radiotracer (e.g., via hybrid bolus-infusion) into the subject 190. In some embodiments, the receipt of the scanning dataset 225 may be in at least partial concurrence with the presentation of the stimuli 220 to the subject 190. In some embodiments, the scanning dataset 225 may identify, define, or otherwise include a set of tomograms 230A-N (hereinafter generally referred to as tomograms 230) acquired by the tomography scanner 170 of the nervous system (e.g., the CNS) of the subject 190 over the time period (T). In some embodiments, the scanning dataset 225 may identify or include raw data (e.g., sinogram data) acquired by the tomography scanner 170 of the nervous system of the subject 190 over the time period (T) to derive, generate, or otherwise define the set of tomograms 230. The set of tomograms 230 may be acquired in accordance with a sampling rate (e.g., ranging between 15-500 Hz) over the time period. Each of the set of tomograms 230 may correspond to an acquisition of the nervous system of the subject 190 at a respective sampling. In some embodiments, each tomogram 230 may be identified or associated with a respective timestamp.

In some embodiments, the image retriever 130 may retrieve, identify, or otherwise receive at least one motion dataset 235 from the motion tracker 175. The motion tracker 175 may produce, create, or otherwise generate the motion dataset 235 to send to the data processing system 105, at least in partial concurrence with the acquisition of the scanning dataset 225 by the tomography scanner 170. The motion dataset 235 may identify or include a set of coordinate measurements of the motion tracker 175 within the scanning volume 208. The coordinate measurements may define or identify at least one of a position (e.g., in x-y-z or polar coordinate system), an orientation, or a movement of the head 202 of the subject 190 over the time period (T) within the scanning volume 208. In some embodiments, the motion tracker 175 may calculate, generate, or otherwise determine the coordinate measurements relative to an electromagnetic source (e.g., an electromagnet or magnet) within the tomography scanner 170.

The electromagnetic source may be situated, arranged, or otherwise disposed on the tomography scanner 170. For example, the motion tracker 175 may instrument the coordinate measurements corresponding to the head 202 of the subject 190 relative to a top portion or a sidewall of the body structure 206 of the tomography scanner 170. The coordinate measurements may have precision ranging between micrometer and centimeter scale, inclusive. The coordinate measurements may be determined over the time period (T) at a sampling rate (e.g., ranging between 15-500 Hz). Concurrent to the acquisition of the scanning dataset 225 or the provision of the stimuli 220, the motion tracker 175 may generate and provide the motion dataset 235 to the data processing system 105.

With the receipt of the scanning dataset 225, the image retriever 130 may extract or identify the set of tomograms 230 from the scanning dataset 225. For example, the scanning dataset 225 may include the set of tomograms 230 generated by the tomography scanner 170. In some embodiments, the image retriever 130 may determine, produce, or otherwise generate the set of tomograms 230 using the scanning dataset 225. For instance, the image retriever 130 may generate the tomograms 230 using the raw scan data (e.g., sinograms) in the scanning dataset 225 in accordance with the imaging modality (e.g., PET or SPECT imaging). With the identification or generation of the set of tomograms 230 from the scanning dataset 225, the image retriever 130 may transform, change, or otherwise modify the set of tomograms 230 in accordance with the motion dataset 235. The modification of the tomograms 230 may be to account for the movement and motion of the head 202 of the subject 190, during the acquisition of the scanning dataset 225 by the tomography scanner 170. In some embodiments, the image retriever 130 may apply motion correction (e.g., using affine alignment or list-mode image correction) to the tomograms 230 using the coordinate measurements of the scanning dataset 225. The motion correction may be applied to the raw scan data or to the tomograms 230 derived from the raw scan data. In some embodiments, the image retriever 130 may generate the tomograms 230 based on the motion (or movement or orientation) of the head 202 as identified in the coordinate measurements of the motion dataset 235 and the scanning dataset 230.

In addition, the signal acquirer 135 executing on the data processing system 105 may retrieve, receive, or otherwise obtain at least one response signal 240 from the one or more sensors 180. The response signal 240 may be associated with at least one physiological function of the subject 190 over the time period. The response signal 240 may be received at least in partial concurrence of the acquisition of the scanning dataset 225 by the tomography scanner 170. The response signal 240 may identify or include a set of measurements associated with the physiological function acquired by a corresponding sensor 180 over the time period (T) at the sampling rate. The measurements associated with the physiological function may include, for example, a heart rate, a heart rate variability, a pupil size, saccade reaction time, power spectral density of current, an event-driven potential of current, skin conductance, skin response, perspiration, respiratory rate, breath duration, and respiratory flow rate, among others. The measurements of the physiological function of the response signal 240 along with imaging of the nervous system in the tomograms 230 may be used to determine conditions in the subject 190 or correlate neurological and physiological functions in the subject 190.

In some embodiments, the signal acquirer 135 may obtain the response signal 240 identifying eye parameters over the time period from the sensor 180 including at least one eye tracker. The eye tracker may be situated, arranged, or otherwise disposed within the headset 165, for example, with the scanner facing towards the eyes of the subject 190. The eye parameters may identify or include, for example, saccade parameters, pupil size, or eye gaze, among others. The signal acquirer 135 may apply computer vision on a video acquired by the eye tracker to determine the eye parameters (e.g., in response to the provision of the stimuli 220 or the performance of the task). In some embodiments, the signal acquirer 135 may obtain the response signal 240 identifying electrocardiogram (ECG) signal over the time period from the sensor 180 including an ECG sensor. The ECG sensor may be situated, arranged, or otherwise disposed on the subject 190 (e.g., about the upper torso or around the heart area). The ECG signal may correspond to an electrical activity of the heart over the time period, and may be used to calculate or determine the heart rate or heart rate variability of the subject 190 (e.g., in response to the provision of the stimuli 220 or the performance of the task). In some embodiments, the signal acquirer 135 may parse the ECG signal to determine the heart rate or heart rate variability of the subject 190.

In some embodiments, the signal acquirer 135 may obtain the response signal 240 identifying respiratory parameters over the time period from the sensor 180 including a respiratory sensor. The respiratory sensor may be a respiratory belt or band, or accelerometer arranged, or otherwise disposed on the subject 190 (e.g., about the upper torso or around the chest area). The respiratory parameters may define or identify respiratory rate, breath duration, and respiratory flow rate, among others. The signal acquirer 135 may parse the response signal 240 to determine the respiratory parameters (e.g., using changes in measurements of chest expansion or contractions) over the time period(e.g., in response to the provision of the stimuli 220 or the performance of the task). In some embodiments, the signal acquirer 135 may obtain the response signal 240 identifying an electroencephalogram (EEG) signal over the time period from the sensor 180 including an EEG sensor. The EEG sensor may be situated, positioned, or arranged over the head 202 of the subject 190. The EEG sensor may be a part of or separate from the headset 165. The EEG signal may identify electrical activity of the nervous system (e.g., the brain) within the head 202 of the subject 190 (e.g., in response to the provision of the stimuli 220 or the performance of the task).

In some embodiments, the signal acquirer 135 may obtain the response signal 240 identifying an electrodermal activity (EDA) signal over the time period from the sensor 180 including the EDA sensor. The EDA sensor (e.g., a galvanic skin response or skin conductance response sensor) may be attached, situated, or otherwise arranged along the skin on the head 202 or the body 204 of the subject 190. The EDA signal may identify measurements of skin conductance or resistance from the subject 190 over the time period (e.g., in response to the provision of the stimuli 220 or the performance of the task). The EDA signal may be used to determine emotion, stress, and nervous system activity in the subject 190. In some embodiments, the signal acquirer 135 may obtain the response signal 240 identifying an electromyography (EMG) signal from the sensor 180 including the EMG sensor. The EMG sensor may be positioned, situated, or otherwise arranged on the head 202 or the body 204 of the subject 190. The EMG may identify electrical activity generated by muscle cells in the subject 190 (e.g., in response to the provision of the stimuli 220 or the performance of the task).

In some embodiments, the signal acquirer 135 may arrange or synchronize the response signal 240 with the set of tomograms 230. To synchronize, the signal acquirer 135 may extract, select, or otherwise identify a set of timestamps corresponding to the set of tomograms 230. In addition, the signal acquirer 135 may extract, select, or otherwise identify a set of timestamps corresponding the response signal 240. Both sets of timestamps may be generated using respective system clocks at the tomography scanner 170 and the sensors 180, substantially corresponding or synchronized (e.g., within 90-95%) with each other. Using the sets of timestamps, the signal acquirer 135 may identify or select at least a portion of the response signal 240 corresponding to the set of tomograms 230. Each measurement in the portion of the response signal 240 may have a timestamp within a margin threshold of a timestamp of the corresponding tomogram 230 in the set. In some embodiments, the signal acquirer 135 may identify or select at least a portion of the set of tomograms 230 corresponding to the response signal 240. Each of the portion of the set of tomograms 230 may have a timestamp within a margin threshold of a timestamp of a corresponding measurement in the response signal 240.

With the provision of the stimuli 220 to the subject 190, the test handler 125 may retrieve, identify, or otherwise receive at least one interaction dataset 245 from the I/O devices 185 over the time period. The interaction dataset 245 may include or identify user interactions by the subject 190 with the I/O devices 185 over the time period (e.g., in response to the provision of the stimuli 220 or the performance of the task). The user interactions may be identified by corresponding timestamps in the interaction dataset 245. The user interactions by the subject 190 may be in furtherance of performing the task as identified in the stimuli 220 provided via the headset 165. The subject 190 may be in the upright posture while interacting with the I/O devices 185 to perform the task. For example, the task provided by the stimuli 220 may be to navigate through a virtual maze, the user interactions by the subject 190 may indicate directions for an avatar to walk through the paths of the virtual maze.

The test handler 125 may produce, determine, or otherwise generate at least one feedback 250 (sometimes herein referred to as a second instruction) to provide to the subject 190. The feedback 250 may be generated based on the task as directed by the stimuli 220 and the user interactions as identified in the interaction dataset 245. For instance, in accordance with the specifications of the task in the instruction 215, the test hander 125 may generate the feedback 250 identifying additional stimuli to be provided to the subject 190. The additional stimuli may indicate whether the subject 190 input the correct response for the task. The feedback 250 may identify or include a visual feedback (e.g., graphics or text), an auditory feedback (e.g., sound or speech), or a haptic feedback (e.g., vibrations), among others, or any combination thereof. With the generation, the test handler 125 may transmit, send, or otherwise provide the feedback 250 to the headset 165. The headset 165 in turn may present the feedback 250 to the subject 190 (e.g., in a similar manner as described above with respect to the stimuli 220). In some embodiments, the functionalities ascribed to the test handler 125 with respect to the interaction dataset 245 and the feedback 250 may be performed by the headset 165.

Referring now to FIG. 18, among others, depicted is a block diagram of a process 300 of determining conditions of nervous systems of subjects in the system 100 for detecting conditions in subjects provided with stimuli. The process 300 may include or correspond to operations in the system 100 to train and use the data analysis model 155. Under the process 300, the model trainer 140 executing on the data processing system 105 may retrieve, obtain, or otherwise identify at least one training dataset 305 for establishing the data analysis model 155. The training dataset 305 may be stored and maintained on the database 160 and may identify or include a set of examples. Each example may identify or include a set of sample tomograms 230′A-N (hereinafter generally referred to as sample tomograms 230′), at least one sample signal 240′, and at least one sample label 310, among others. The sample tomograms 230′ may be similar to the set of tomograms 230 and may be of a nervous system of another subject. The sample signal 240′ may be associated with the physiological function of the subject (e.g., same as the tomogram 230′). The response signal 240′ may be synchronized with the sample tomograms 230′ (e.g., with corresponding timestamps).

The sample label 310 may identify at least one of a set of conditions in the nervous system of the sample subject. The set of conditions may, for example, include a tumor (e.g., presence, absence, or severity), brain injury (e.g., presence, absence, or severity), epilepsy (e.g., presence, absence, or severity), type of neurodegenerative disease (e.g., Alzheimer, MCI, or Huntington's disease), stroke (e.g., ischemic or hemorrhagic, presence, absence, or severity), a biomarker (e.g., related to disease or disease), disease response (e.g., effective or ineffective), drug response (e.g., presence, absence, or severity), movement disorder (e.g., Parkinson's, tremor, dystonia), or behavioral condition (e.g., presence, absence, or severity), among others. In some embodiments, the sample label 310 may also identify a correlation between a portion (e.g., a processing region) within the nervous system of the sample subject and the condition. When the response signal 240 identifies the eye parameters, the sample label 310 may identify or determine the condition in the portion of the nervous system associated with the visual or eye functionality of the subject 190. The condition may be, for example, at least one of a dorsal raphe, a locus coeruleus, a superior colliculus, a basal ganglia, or a frontal cortex within the nervous system, among others. When the response signal 240 identifies the ECG signal, the sample label 310 may identify or determine the condition associated with the parasympathetic system in the subject 190. The condition may include, for instance, at least one of a cognitive function, an emotional regulation, or stress management, among others.

The model trainer 140 may initialize, train, or establish the data analysis model 155 using the training dataset 305. In some embodiments, the data analysis model 155 may be initialized, trained, and established using the training dataset 305 (e.g., by a computing system different from the data processing system 105). When implemented using a machine learning model, the data analysis model 155 may be trained in accordance with any learning techniques, such as supervised learning, unsupervised learning, Q-learning or weakly supervised learning, among others. When implemented using a statistical model, the data analysis model 155 may be established using data fitting techniques (e.g., using a loss metric). With the identification, the model trainer 140 may feed or apply the set of sample tomograms 230′ and the sample signal 240′ from each example of the training dataset 305 to the data analysis model 155. To apply, the model trainer 140 may process the input set of sample tomograms 230′ and the sample signal 240′ in accordance with the set of weights of the data analysis model 155.

From processing the input, the model trainer 140 may produce, determine, or otherwise generate an output identifying at least one of the conditions in the nervous system in the sample subject. The model trainer 140 may compare the output with the corresponding condition in the sample label 310. Based on the comparison, the model trainer 140 may calculate, generate, or determine a loss metric. The loss metric may be in accordance with a loss function (e.g., a hinge loss, a mean squared error (MSE), a mean absolute error (MAE), a cross-entropy loss, a Huber loss, or a log loss). Using the loss metric, the model trainer 140 may modify or update one or more of the set of weights of the data analysis model 155. This process can be iteratively repeated until the data analysis model 155 reaches a convergence condition to stop or cease the training process. When implemented using a statistical model, the data analysis model 155 may be established using data fitting techniques (e.g., using a loss metric).

The model applier 145 executing on the data processing system 105 may feed or apply the set of tomograms 230 and the response signal 240 to the data analysis model 155. In applying, the model applier 145 may feed, input, or otherwise provide the set of tomograms 230 and the response signal 240 to the data analysis model 155. In some embodiments, the model applier 145 may provide the set of tomograms 230 and the identified corresponding portion of the response signal 240 to the data analysis model 155. In some embodiments, the model applier 145 may provide the corresponding portion of the set of tomograms 230 and the response signal 240 to the data analysis model 155. The model applier 145 may process the set of tomograms 230 and the response signal 240 in accordance with the set of weights of the data analysis model 155.

From processing, the model applier 145 may produce, generate, or otherwise determine at least one condition identifier 315 associated with the nervous system of the subject 190. The condition identifier 315 may define or indicate the condition in the nervous system of the subject 190. The condition identifier 315 may identify or include, for example: a tumor (e.g., presence, absence, or severity), brain injury (e.g., presence, absence, or severity), epilepsy (e.g., presence, absence, or severity), type of neurodegenerative disease (e.g., Alzheimer, mild cognitive impairment (MCI), or Huntington's disease), stroke (e.g., ischemic or hemorrhagic, presence, absence, or severity), a biomarker (e.g., related to disease or disease), disease response (e.g., effective or ineffective), drug response (e.g., presence, absence, or severity), movement disorder (e.g., Parkinson's, tremor, dystonia), or behavioral condition (e.g., presence, absence, or severity), among others. In some embodiments, the model applier 145 may generate multiple condition identifiers 310, from processing the input tomograms 230 and response signal 240 with the data analysis model 155.

In some embodiments, the model applier 145 may determine the condition identifier 315 by applying the data analysis model 155, based on a type of measurements in the response signal 240 or the sensors 180 used to generate the response signal 240. The condition identifier 315 may identify or define a correlation (e.g., in terms of Pearson correlation coefficient, Spearman's rank correlation, or covariance) between a portion (e.g., a processing region) within the nervous system of the subject 190 and the condition. In some embodiments, the condition identifier 315 may identify or define a correlation between a portion (e.g., a processing region) within the nervous system of the subject 190 and the physiological function of the subject 190.

When the response signal 240 identifies the eye parameters, the model applier 145 may determine the condition identifier 315 may identify or determine the condition in the portion of the nervous system associated with the visual or eye functionality of the subject 190. The condition may be, for example, at least one of a dorsal raphe, a locus coeruleus, a superior colliculus, a basal ganglia, or a frontal cortex within the nervous system, among others. When the response signal 240 identifies the ECG signal, the model applier 145 may identify or determine the condition associated with the parasympathetic system in the subject 190. The condition may include, for instance, at least one of a cognitive function, an emotional regulation, or stress management, among others.

The output handler 150 executing on the data processing system 105 may store and maintain an association between the subject 190 (e.g., or an identifier for the subject 190) and the condition identifier 315 on the database 160. The association may be among one or more of: the subject 190, acquired data (e.g., the scanning dataset 225, the set of tomograms 230, the motion dataset 235, the response signal 240, and the interaction dataset 245), and instruction 215 (including an identification of the stimuli 220), among others. The association may be stored and maintained using one or more data structures. The data structures may include, for example, arrays, matrixes, tables, linked lists, stacks, queues, trees, tables, or heaps, among others. The data structures may be written, stored, and maintained on the database 160 using one or more files, such as database format file (e.g., structured query language (SQL) or comma-separated values (CSV)) or a markup file (e.g., an extensible markup language (XML) or JavaScript Objection Notation (JSON)), among others.

The output handler 150 may produce, create, or otherwise generate at least one output 320 based on the condition identifier 315. In some embodiments, the output 320 may be generated based on one or more of the set of tomograms 230, the response signal 240, and the association between the subject 190 and the condition identifier 315, among others. With the generation, the output handler 150 may send, provide, or transmit the output 320 for presentation of the display 115. The output handler 150 may send, transmit, or otherwise provide information based on the output 320 for presentation via the display 115. The information may include, for example, the condition identifier 315, the acquired data (e.g., the scanning dataset 225, the set of tomograms 230, the motion dataset 235, the response signal 240, and the interaction dataset 245), and the instruction 215 (including an identification of the stimuli 220), among others.

The display 115 (or a computing device connected thereto) may display, render, or otherwise present the information using the output 320 from the data processing system 105.

The information may be presented to the clinician examining the subject 190 to evaluate the conditions associated with the nervous system or the physiological function (or both) in the subject 190. For example, the display 115 may present at least one of the tomograms 230 depicting the brain of the subject 190, along with one or more response signals 240 and the condition identifier 315. The information may be used by the clinician to diagnose the condition associated with the nervous system in the subject 190. The information may also be used by the clinician to associate or correlate particular conditions in the nervous system with one or more physiological functions in the subject 190.

In this manner, the data processing system 105 may provide for stimuli to direct the subject 190 to perform certain tasks and receive feedback for interactions in furtherance of the task, all while in a natural, upright posture. With the tomography device 170 used to generate the tomograms 230 also allowing for the subject 190 to be in the upright posture, the tomography scanner 170 may acquire tomograms 230 of the nervous system with behaviors. The tomograms 230 may thus be different from the subject 190 being in a prone or supine position, and may be able to capture brain activity that would otherwise not been possible to acquire with the subject 190 in the prone or supine position. The acquisition of tomograms 230 from the tomography scanner 170 may thus be an improvement to imaging brain activity, relative to approaches that rely on the subject 190 being in prone or supine position.

Furthermore, the data processing system 105 may in partial concurrence aggregate the tomograms 230 acquire by the tomography scanner 170 and the response signals 240 of various physiological functions of the subject 190, thereby providing for a centralized platform to process data in multiple modalities. The data processing system 105 may also rely on the motion tracker 175 to identify precise coordinate measurements regarding the movements of the head 202 within the scanning volume 208 of the tomography scanner 170, and to compensate such movements within the tomograms 230 acquired of the brain of the subject 190. The data processing system 105 may thus be able to reconstruct or recover better quality tomograms 230 from the scanning dataset 225. The combination and processing of multimodal data using the data analysis model 155 may greatly enhance the functionalities of the data processing system 105, compared to more restrictive approaches that rely on separate devices to process each modality.

Referring now to FIG. 20, depicted is a flow diagram of a method 400 of detecting conditions in subjects provided with stimuli. The method 400 may be performed by or implemented using the system 100 described herein in conjunction with FIGS. 22-24 or the system 500 detailed herein in Section D. Under the method 400, a computing system may send an instruction for a headset to provide a stimuli to a subject (405). The computing system may receive a dataset defining a set of tomograms from a tomography scanner (410). The computing system may receive a set of motion measurements from a motion tracker (415). The computing system may obtain a response signal from a sensor (420). The computing system may apply a machine learning (ML) model to the set of tomograms and the response signal (425). The computing system may determine a condition of a nervous system of the subject based on applying the ML model (430). The computing system may generate an output based on the determined condition (435).

### D. Computing and Network Environment

Various operations described herein can be implemented on computer systems. FIG. 21 shows a simplified block diagram of a representative server system 500, client computing system 514, and network 526 usable to implement certain embodiments of the present disclosure. In various embodiments, server system 500 or similar systems can implement services or servers described herein or portions thereof. Client computing system 514 or similar systems can implement clients described herein. The system 500 described herein can be similar to the server system 500. Server system 500 can have a modular design that incorporates a number of modules 502 (e.g., blades in a blade server embodiment); while two modules 502 are shown, any number can be provided. Each module 502 can include processing unit(s) 504 and local storage 506.

Processing unit(s) 504 can include a single processor, which can have one or more cores, or multiple processors. In some embodiments, processing unit(s) 504 can include a general-purpose primary processor as well as one or more special-purpose co-processors such as graphics processors, digital signal processors, or the like. In some embodiments, some or all processing units 504 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 504 can execute instructions stored in local storage 506. Any type of processors in any combination can be included in processing unit(s) 504.

Local storage 506 can include volatile storage media (e.g., DRAM, SRAM, SDRAM, or the like) and/or non-volatile storage media (e.g., magnetic or optical disk, flash memory, or the like). Storage media incorporated in local storage 506 can be fixed, removable or upgradeable as desired. Local storage 506 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 504 need at runtime. The ROM can store static data and instructions that are needed by processing unit(s) 504. The permanent storage device can be a non-volatile read-and-write memory device that can store instructions and data even when module 502 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 506 can store one or more software programs to be executed by processing unit(s) 504, such as an operating system and/or programs implementing various server functions such as functions of the system 500 of FIG. 5 or any other system described herein, or any other server(s) associated with system 500 or any other system described herein.

“Software” refers generally to sequences of instructions that, when executed by processing unit(s) 504 can cause server system 500 (or portions thereof) to perform various operations, thus, defining one or more specific machine embodiments that execute and perform the operations of the software programs. The instructions can be stored as firmware residing in read-only memory and/or program code stored in non-volatile storage media that can be read into volatile working memory for execution by processing unit(s) 504. Software can be implemented as a single program or a collection of separate programs or program modules that interact as desired. From local storage 506 (or non-local storage described below), processing unit(s) 504 can retrieve program instructions to execute and data to process in order to execute various operations described above.

In some server systems 500, multiple modules 502 can be interconnected via a bus or other interconnect 508, forming a local area network that supports communication between modules 502 and other components of server system 500. Interconnect 508 can be implemented using various technologies including server racks, hubs, routers, etc.

A wide area network (WAN) interface 510 can provide data communication capability between the local area network (interconnect 508) and the network 526, such as the Internet. Technologies can be used, including wired (e.g., Ethernet, IEEE 502.3 standards) and/or wireless technologies (e.g., Wi-Fi, IEEE 502.24 standards).

In some embodiments, local storage 506 is intended to provide working memory for processing unit(s) 504, providing fast access to programs and/or data to be processed while reducing traffic on interconnect 508. Storage for larger quantities of data can be provided on the local area network by one or more mass storage subsystems 512 that can be connected to interconnect 508. Mass storage subsystem 512 can be based on magnetic, optical, semiconductor, or other data storage media. Direct-attached storage, storage area networks, network-attached storage, and the like can be used. Any data stores or other collections of data described herein as being produced, consumed, or maintained by a service or server can be stored in mass storage subsystem 512. In some embodiments, additional data storage resources may be accessible via WAN interface 510 (potentially with increased latency).

Server system 500 can operate in response to requests received via WAN interface 510. For example, one of the modules 502 can implement a supervisory function and assign discrete tasks to other modules 502 in response to received requests. Work allocation techniques can be used. As requests are processed, results can be returned to the requester via WAN interface 510. Such operation can generally be automated. Further, in some embodiments, WAN interface 510 can connect multiple server systems 500 to each other, providing scalable systems capable of managing high volumes of activity. Other techniques for managing server systems and server farms (collections of server systems that cooperate) can be used, including dynamic resource allocation and reallocation.

Server system 500 can interact with various user-owned or user-operated devices via a wide area network such as the Internet. An example of a user-operated device is shown in FIG. 5 as client computing system 514. Client computing system 514 can be implemented, for example, as a consumer device such as a smartphone, other mobile phone, tablet computer, wearable computing device (e.g., smart watch, eyeglasses), desktop computer, laptop computer, and so on.

For example, client computing system 514 can communicate via WAN interface 510. Client computing system 514 can include computer components such as processing unit(s) 516, storage device 518, network interface 520, user input device 522, and user output device 524. Client computing system 514 can be a computing device implemented in a variety of form factors, such as a desktop computer, laptop computer, tablet computer, smartphone, other mobile computing device, wearable computing device, or the like.

Processing unit(s) 516 and storage device 518 can be similar to processing unit(s) 504 and local storage 506 described above. Suitable devices can be selected based on the demands to be placed on client computing system 514; for example, client computing system 514 can be implemented as a “thin” client with limited processing capability or as a high-powered computing device. Client computing system 514 can be provisioned with program code executable by processing unit(s) 516 to enable various interactions with server system 500.

Network interface 520 can provide a connection to the network 526, such as a wide area network (e.g., the Internet) to which WAN interface 510 of server system 500 is also connected. In various embodiments, network interface 520 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 522 can include any device (or devices) via which a user can provide signals to client computing system 514; client computing system 514 can interpret the signals as indicative of particular user requests or information. In various embodiments, user input device 522 can include any or all of a keyboard, touch pad, touch screen, mouse or other pointing device, scroll wheel, click wheel, dial, button, switch, keypad, microphone, and so on.

User output device 524 can include any device via which client computing system 514 can provide information to a user. For example, user output device 524 can include a display-to-display images generated by or delivered to client computing system 514. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 524 can be provided in addition to or instead of a display. Examples include indicator lights, speakers, tactile “display” devices, printers, and so on.

Some embodiments include electronic components, such as microprocessors, storage and memory that store computer program instructions in a computer-readable storage medium. Many of the features described in this specification can be implemented as processes that are specified as a set of program instructions encoded on a computer-readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 504 and 516 can provide various functionality for server system 500 and client computing system 514, including any of the functionality described herein as being performed by a server or client, or other functionality.

It will be appreciated that server system 500 and client computing system 514 are illustrative and that variations and modifications are possible. Computer systems used in connection with embodiments of the present disclosure can have other capabilities not specifically described here. Further, while server system 500 and client computing system 514 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies including but not limited to the specific examples described herein. Embodiments of the present disclosure can be realized using any combination of dedicated components and/or programmable processors and/or other programmable devices. The various processes described herein can be implemented on the same processor or different processors in any combination.

Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer-readable storage media; suitable media include magnetic disk or tape, optical storage media such as compact disk (CD) or DVD (digital versatile disk), flash memory, and other non-transitory media. Computer-readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method of detecting conditions in subjects provided with stimuli, comprising:
sending, by one or more processors, to a headset situated on a head of a first subject in an upright posture, an instruction for the headset to present a stimuli to direct the subject to perform a task over a portion of a time period;
receiving, by the one or more processors, via a tomography scanner arranged about the head of the first subject in the upright posture, a dataset defining a first plurality of tomograms of a first nervous system of the first subject over the time period;
obtaining, by the one or more processors, via a sensor, a first signal associated with a first physiological function of the first subject over the time period, in at least partial concurrence of acquisition of the dataset defining the first plurality of tomograms via the tomography scanner;
applying, by the one or more processors, a model to the first plurality of tomograms and the first response signal, the model established using a plurality of examples, each of the plurality of examples identifying: (i) a respective plurality of second tomograms of a respective second nervous system of a respective second subject, (ii) a respective second signal associated with a respective second physiological function of the respective second subject, and (iii) a respective condition of a plurality of conditions identified for the respective second nervous system of the respective second subject;
determining, by the one or more processors, from the plurality of conditions, a condition associated with the first nervous system of the first subject based on applying the model to the first plurality of tomograms and the first signal; and
storing, by the one or more processors, using one or more data structures, an association between the condition and the first subject.

2. The method of claim 1, further comprising:
receiving, by the one or more processors, a plurality of user interactions by the subject while in the upright posture from an input/output (I/O) device in response to performing the task; and
sending, by the one or more processors, a second instruction including feedback to provide to the subject, based on the task and plurality of interactions, the feedback including at least one of a visual feedback, an auditory feedback, or a haptic feedback.

3. The method of claim 1, further comprising:
generating, by the one or more processors, an output based on at least one of the first plurality of tomograms, the first signal, or the association between the condition and the first subject; and
providing, by the one or more processors, to a display for presentation, information using the output.

4. The method of claim 1, further comprising:
receiving, by the one or more processors, from the sensor including a motion tracker situated on the headset, a plurality of coordinate measurements identifying motion of the head over the time period, wherein the motion tracker is further configured to determine the plurality of coordinate measurements relative to an electromagnetic source within the tomography scanner; and
generating, by the one or more processors, the first plurality of tomographs based on the motion of head identified in the plurality of coordinate measurements and the dataset received via the tomography scanner.

5. The method of claim 1, further comprising identifying, by the one or more processors, at least a portion of the first signal corresponding to the first plurality of tomographs, using a first plurality of timestamps of the first plurality of tomographs and a second plurality of timestamps of the first signal; and
wherein applying the model further comprises applying the model to the first plurality of tomographs and at least the portion of the first signal
wherein the model comprises at least one of a machine learning model or a statistical model.

6. The method of claim 1, wherein sending the instruction further comprises sending the instruction for the headset to present, via a display and a loudspeaker, an audiovisual stimuli to direct the first subject to perform the task including at least one of a cognitive task or a behavior task via an I/O device, and
wherein receiving the dataset further comprises receiving the dataset defining the first plurality of tomograms subsequent to injection of a hybrid bolus-infusion radiotracer into the first subject.

7. The method of claim 1, wherein obtaining the first signal further comprises obtaining, from the sensor including an eye tracker on the headset, the first signal to identify at least one of a saccade parameters or pupil size of eyes of the first subject over the time period, and
wherein determining the condition further comprises determining the condition from the plurality of conditions associated with at least one of a dorsal raphe, a locus coeruleus, a superior colliculus, a basal ganglia, or a frontal cortex within the first nervous system of the first subject.

8. The method of claim 1, wherein obtaining the first signal further comprises obtaining, from the sensor including an electrocardiogram (ECG) sensor positioned on the first subject, the first signal including an ECG signal over the time period, and
wherein determining the condition further comprises determining the condition from the plurality of conditions associated with at least one of a parasympathetic system in the first nervous system of the first subject, the plurality of conditions comprises at least one of a cognitive function, an emotional regulation, or a stress management.

9. The method of claim 1, wherein obtaining the first signal further comprises obtaining the first signal at least one of: (i) respiration sensor, (ii) an electroencephalogram (EEG) sensor, (iii) an electrodermal activity (EDA) sensor, or (iv) an electromyography (EMG) sensor.

10. The method of claim 1, wherein the tomography scanner comprises a body structure defining a scanning volume within which the head of the first subject is situated, wherein the tomography scanner is configured to permit the head of the first subject with a degree of movement within the scanning volume, wherein the tomography scanner is situated on a mobile structure.

11. A system for detecting conditions in subjects provided with stimuli, comprising:
one or more processors coupled with memory, configured to:
send, to a headset situated on a head of a first subject in an upright posture, an instruction for the headset to present a stimuli to direct the subject to perform a task over a portion of a time period;
receive, via a tomography scanner arranged about the head of the first subject in the upright posture, a dataset defining a first plurality of tomograms of a first nervous system of the first subject over the time period;
obtain, via a sensor, a first signal associated with a first physiological function of the first subject over the time period, in at least partial concurrence of acquisition of the dataset defining the first plurality of tomograms via the tomography scanner;
apply a model to the first plurality of tomograms and the first response signal, the model established using a plurality of examples, each of the plurality of examples identifying: (i) a respective plurality of second tomograms of a respective second nervous system of a respective second subject, (ii) a respective second signal associated with a respective second physiological function of the respective second subject, and (iii) a respective condition of a plurality of conditions identified for the respective second nervous system of the respective second subject;
determine, from the plurality of conditions, a condition associated with the first nervous system of the first subject based on applying the model to the first plurality of tomograms and the first signal; and
store, using one or more data structures, an association between the condition and the first subject.

12. The system of claim 11, wherein the one or more processors are further configured to:
receive a plurality of user interactions by the subject while in the upright posture from an input/output (I/O) device in response to performing the task; and
send a second instruction including feedback to provide to the subject, based on the task and plurality of interactions, the feedback including at least one of a visual feedback, an auditory feedback, or a haptic feedback.

13. The system of claim 11, wherein the one or more processors are further configured to:
generate an output based on at least one of the first plurality of tomograms, the first signal, or the association between the condition and the first subject; and
provide, to a display for presentation, information using the output.

14. The system of claim 11, wherein the one or more processors are further configured to:
receive, from the sensor including a motion tracker situated on the headset, a plurality of coordinate measurements identifying motion of the head over the time period, wherein the motion tracker is further configured to determine the plurality of coordinate measurements relative to an electromagnetic source within the tomography scanner; and
generate the first plurality of tomographs based on the motion of head identified in the plurality of coordinate measurements and the dataset received via the tomography scanner.

15. The system of claim 11, wherein the one or more processors are further configured to:
identify at least a portion of the first signal corresponding to the first plurality of tomographs, using a first plurality of timestamps of the first plurality of tomographs and a second plurality of timestamps of the first signal; and
apply the model to the first plurality of tomographs and at least the portion of the first signal, wherein the model comprises at least one of a machine learning model or a statistical model.

16. The system of claim 11, wherein the one or more processors are further configured to:
send the instruction for the headset to present, via a display and a loudspeaker, an audiovisual stimuli to direct the first subject to perform the task including at least one of a cognitive task or a behavior task via an I/O device, and
receive the dataset defining the first plurality of tomograms subsequent to injection of a hybrid bolus-infusion radiotracer into the first subject.

17. The system of claim 11, wherein the one or more processors are further configured to:
obtain, from the sensor including an eye tracker on the headset, the first signal to identify at least one of a saccade parameters or pupil size of eyes of the first subject over the time period, and
determine the condition from the plurality of conditions associated with at least one of a dorsal raphe, a locus coeruleus, a superior colliculus, a basal ganglia, or a frontal cortex within the first nervous system of the first subject.

18. The system of claim 11, wherein the one or more processors are further configured to:
obtain, from the sensor including an electrocardiogram (ECG) sensor positioned on the first subject, the first signal including an ECG signal over the time period, and
determine the condition from the plurality of conditions associated with at least one of a parasympathetic system in the first nervous system of the first subject, the plurality of conditions comprises at least one of a cognitive function, an emotional regulation, or a stress management.

19. The system of claim 11, wherein the one or more processors are further configured to obtain the first signal at least one of: (i) respiration sensor, (ii) an electroencephalogram (EEG) sensor, (iii) an electrodermal activity (EDA) sensor, or (iv) an electromyography (EMG) sensor.

20. The system of claim 11, wherein the tomography scanner comprises a body structure defining a scanning volume within which the head of the first subject is situated, wherein the tomography scanner is configured to permit the head of the first subject with a degree of movement within the scanning volume, wherein the tomography scanner is situated on a mobile structure.
